SEQUENCE LISTING (4 sequences)

SEQ ID NO: 1 - protein, 175 aa, Homo sapiens
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 2 - DNA, 531 bp, Homo sapiens
  CDS (1)..(525)
atgactccattaggtcctgcttcttctctgccgcaaagctttctgctgaaatgtctggaacaggttcgtaaaatccagggtgacggtgctgcactgcaagaaaaactgtgcgctacttacaaactgtgccatccggaagagctggtactgctgggtcattctcttgggatcccgtgggctccgctgtcttcttgtccatctcaagctcttcagctggctggttgtctgtctcaactgcattctggtctgttcctgtatcagggtcttctgcaagctctggaaggtatctctccggaactgggtccgactctggacactctgcagctagatgtagctgactttgctactactatttggcaacagatggaagagctcggtatggcaccagctctgcaaccgactcaaggtgctatgccggcattcgcttctgcattccagcgtcgtgcaggaggtgtactggttgcttctcatctgcaatctttcctggaagtatcttaccgtgttctgcgtcatctggctcagccgtaatag

SEQ ID NO: 3 - protein, 207 aa, Homo sapiens
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 4 - protein, 204 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

(19) United States
(12) Patent Application Publication
(10) Pub. No.: US 2016/0361426 A1
(43) Pub. Date: Dec. 15, 2016

(54) PEGYLATED GRANULOCYTE COLONY STIMULATING FACTOR (GCSF)

(71) Applicant: Prolong Pharmaceuticals, LLC, South Plainview, NJ (US)

(72) Inventors: Abraham Abuchowski, Califon, NJ (US); Ronald G. Jubin, Watchung, NJ (US); Peter J. Buontempo, Westfield, NJ (US); Friedericke Kazo, New Ipswich, NH (US)

(21) Appl. No.: 15/181,333

(22) Filed: Jun. 13, 2016

Related U.S. Application Data
(60) Provisional application No. 62/174,373, filed on Jun. 11, 2015.
(60) Provisional application No. 62/184,042, filed on Jun. 24, 2015.

(51) Int. Cl. A61K 47/48; A61K 38/19

(52) U.S. Cl. CPC A61K 47/48215; A61K 38/193

(57) ABSTRACT

This invention relates to novel PEGx-GCSF conjugates, wherein x is the amount of PEG per GCSF and ranges from 4 to 8. The invention also relates to PEG[x]-GCSF populations of individual PEGx-GCSF conjugates, wherein [x] is the average amount of PEG per GCSF of the population and is 4 or greater. The inventive compositions have unexpected therapeutic efficacy, while avoiding or substantially reducing the likelihood of adverse side effects.

### RELATED APPLICATION

This invention claims priority to provisional patent applications U.S. Ser. No. 62/174,373 filed on Jun. 11, 2015, and U.S. Ser. No. 62/184,042 filed Jun. 24, 2015, the entire contents of which are both incorporated herein by reference.

### FIELD OF THE INVENTION

This invention relates to novel PEG-GCSF conjugates having unexpected therapeutic efficacy, while avoiding or substantially reducing the likelihood of adverse side effects.

### BACKGROUND

In recent years, non-antigenic water-soluble polymers, such as polyethylene glycol (“PEG”), have been used for the covalent modification of polypeptides of therapeutic and diagnostic importance. PEG is a polymer that is nontoxic, nonimmunogenic, highly water soluble, and readily cleared from the body. PEG has many applications and is commonly used in foods, cosmetics, beverages, and prescription medicines. Pharmaceutical grade PEGs are approved for use in the United States by the FDA and are widely used as biopharmaceutical carriers, given their high degree of biocompatibility. PEGylation can modify certain characteristics of biopharmaceuticals without altering their function, thereby enhancing the therapeutic effect.

Neutrophil granulocytes are the most abundant type of white blood cells in mammals, and comprise an essential part of the innate immune system. Their production is regulated via granulocyte colony stimulating factor (GCSF) engagement to its cognate receptor located on the surface of CD34+ myeloid precursor cells. Receptor engagement results in receptor chain oligomerization, rearrangement and signal transduction mediated through intracellular kinases, resulting in gene expression patterns promoting differentiation and cell division, thereby increasing neutrophil counts. The importance of GCSF receptor signaling is exemplified in individuals with inborn genetic errors in the cytokine:receptor signaling pathway. Collectively, limited signaling will result in a reduced ability to maintain appropriate levels of neutrophils. Signaling via the GCSF receptor is important for the production and maintenance of neutrophils, and individuals with inborn genetic errors in GCSF signaling have reduced neutrophil counts and therefore are predisposed to serious and recurrent microbial infections.

Similarly, many cancer therapies exhibit potent inhibition of neutrophil levels due to their anti-proliferative activity. One of the most serious potential side effects of many types of chemotherapy drugs is a low white blood cell count that includes decreased neutrophil levels (neutropenia). Neutropenia can put some patients at risk for severe infections and therefore may force cessation of chemotherapy treatment cycle. In fact, complications associated with a low white blood cell count are the most common causes of dose reductions or delays in chemotherapy (see Link, et al. (2001) Cancer 92:1354-1367; Lyman, et al. (2003) J. Clin. Oncol. 21:4524-4531; and Lyman, et al. (2002) Am. J. Med. 112:406-411, the entirety of each of which are incorporated herein by reference). This dose-dependent phenomenon has dramatically limited the therapeutic dosages of many oncology drugs.

The development of recombinant GCSF (filgrastim) for clinical use has led to dramatic improvement of both individuals born with severe chronic neutropenia (SCN) as well as those undergoing cancer therapies with potent anti-neutrophil activity. GCSF is a small protein that is readily removed through the renal system. The mouse version of GCSF was purified from explanted tissues in 1983, and the human equivalent purified from a cancer cell line grown in culture inadvertently expressing GCSF in high concentrations in 1985 (see e.g. Welte, et al. (1985) PNAS USA 82:1526-30, which is incorporated herein by reference in its entirety). The human GCSF was found to be a glycoprotein around 19 kD which was variably acidic depending on the carbohydrate component. It was later found that the carbohydrate component was optional for biologic activity. The cloning and characterization of human recombinant GCSF took place between 1984 and 1986, and led to its expression in E. coli cells and eventually to human clinical trials testing the compound in patients suffering from chemotherapy-induced neutropenia. In 1991, recombinant human GCSF made in E. coli was approved by the U.S. FDA for this use (named Filgrastim, trade-named Neupogen®), and in 1993 a related Chinese hamster ovary cell expressed form was approved in Europe (under the name lenograstim). It was found that the core protein included 174 amino acids, although multiple variants are known to exist (see e.g. Ngata, et al. (1986) Nature 319:415-18; Souza, et al. (1986) Science 232:61-5; U.S. Pat. No. 4,999,291, each of which are incorporated herein by reference).

U.S. Pat. Nos. 4,810,643, 4,999,291, 5,582,823 and 5,580,755, assigned to Amgen, Inc. and claiming priority back to U.S. patent application Ser. 07/768,959, filed Aug. 23, 1985, provide certain human pluripotent GCSF molecules and methods of their production, each of which are incorporated herein by reference in their entirety. These molecules form the basis for the approved Neupogen product. There is no discussion of potential PEGylation of the molecule in these cases.

Because Filgrastim is readily degraded in vivo, Neupogen requires daily administration during an incidence of febrile neutropenia brought on by cancer treatments. However, PEGylation represents a plausible approach to increasing the hydrodynamic radius of the GCSF protein, reducing serum clearance and promoting drug half-life in vivo. Using site-specific PEGylation at the N-terminus of GCSF with aldehyde-activated, 20 kDa linear PEG (see PCT Publication No. WO 96/11953 as well as U.S. Pat. Nos. 5,824,784 and 7,090,835), PEG-Filgrastim was developed, and was approved by the U.S. FDA in 2002 under the tradename NEULASTA® (NEULASTA® [package insert]. Thousand Oaks, Calif., Amgen, Inc., revised 02/2010; NEULASTA® [package insert]. Thousand Oaks, Calif., Amgen, Inc., revised 4/2016 v1, both revisions incorporated herein by reference). This mono-PEGylated version of GCSF, with the PEG moiety covalently attached to the amino terminus of the protein, increases the molecular weight of the GCSF protein, greatly reducing renal clearance. The location of the PEG group at the amino terminus is not particularly disruptive to the GCSF protein—GCSF receptor interaction, since the protein residues in the binding region involved in receptor interaction are not directly PEGylated or sterically hindered by the amino terminal 20 kDa PEG.

Several alternate strategies for providing a stabilized GCSF molecule also have been proposed. Linking PEG to a cysteine residue has provided certain improvements in targeting. Thiol reactive PEGs (including PEG-maleimide) have been linked to GCSF at its free cysteine residue. Veronese, et al. (2007) Bioconjugate Chem. 18:1824-1830 described the PEGylation of GCSF at Cys18, which was shown to increase aggregation although the aggregates were not covalently aggregated. Similarly, Hao, et al. (2006) Biodrugs 20:357-363 described the conjugation of PEG-maleimide to Cys18, which was shown to increase the half life of the molecule.

Site-specific mutagenesis is a further approach which has been used to prepare polypeptides for site-specific polymer attachment. For example, U.S. Pat. No. 6,646,110 describes polypeptide conjugates that exhibit GCSF activity and have an amino acid residue that comprise an attachment group for a PEG or oligosaccharide moiety inserted. These can include lysine, glutamic acid, cysteine or aspartic acid.

WO 2011/041376 reflects yet another approach to site-specific PEGylation, by one of the inventors of the instant application. The contents of WO 2011/041376 are incorporated by reference in its entirety. In this earlier work, methoxy-PEG acetaldehyde was reacted with GCSF in a DMSO-containing reaction buffer to yield a population of monoPEGylated GCSF conjugates, wherein the conjugation is at a lysine group near the N-terminus, and wherein at least 30% of the composition is not N-terminally PEGylated. In an alternate embodiment, the composition comprised at least 80% monoPEGylated GCSF conjugate, wherein at least 30% of the composition is not N-terminally PEGylated.

As an alternative to site-specific PEGylation, random PEGylation using N-hydroxy-succinimide esters forms stable protein-PEG conjugates via amide bonds. These ester reagents are relatively specific for the reaction with amino groups of the lysine residues and the N-terminus, but react to minor degrees also with other protein nucleophiles like histidine, serine and tyrosine residues. Reaction conditions like temperature, pH, amount of PEG reagent, and time define the heterogeneity of the product (i.e., mono-, di-, tri- and higher-PEGylated conjugates can be formed). Due to reactions with different nucleophilic groups on the protein, multi-PEGylated (and even mono-PEGylated) conjugates yield positional isomers that can differ substantially in their biological and biomedical properties. The high degree of PEGylation variability, as well as the capability to manufacture in a reproducible manner, has limited the use of SC-PEG in clinical drug development. However there are examples (e.g., Oncaspar, Adagen) that demonstrate such conjugates can be clinically relevant in some situations.

Prior attempts to employ amine-reactive PEGs to form PEG-GCSF by attaching at exposed amine groups on lysine residues and N-terminal amino acids have been reported with limited success. It was observed that such an approach is not optimal for GCSF because the protein contains four lysine residues and an N-terminal amino acid with the lysine residues located in receptor binding regions. Modification of GCSF with amine-reactive PEG reagents therefore reduces in vitro biological activity of the protein by 3- to 50-fold, depending on the number and sizes of attached PEG molecules. Loss of in vitro bioactivity is greatest when GCSF is modified with large PEGs, e.g., 20 kDa PEGs, which are most useful in extending the protein's half-life. Amine-PEGylated GCSF is heterogeneous, occurring as a complex mixture of at least four isoforms and multiple molecular weight species, all of which may have different specific activities.

A particular example of this approach is described in two journal articles from the early 1990s, by a group of pharmaceutical investigators at the Kirin Brewery Company: Tanaka et al. (1991) Cancer Research 51:3710-3714 and Satake-Ishikawa et al. (1992) Cell Structure and Function 17:157-160. These investigators prepared mixtures of conjugates wherein each molecule of the GCSF protein apparently was modified by one, two, or three PEGs, with an average of two. The activated PEG reagent utilized by these investigators was SS-PEG (4.5 kDa or 10 kDa). Although the resulting amide bond between the protein and PEG is stable, the linker contains an ester group which is hydrolytically labile. Such hydrolysis will occur as long as the compound is in an aqueous medium and, therefore, the PEG number continuously decreases as long as it is in solution. Hydrolysis of the ester linkage leaves behind a succinate group which can cyclize to a succinimidyl group. Such non-natural residues can potentially result in antibody responses, including immunogenicity.

Side effects associated with known versions of GCSF, including PEGylated versions, include dose-related glomerulonephritis and adverse and serious adverse events of bone pain. This has resulted in many cancer patients suffering through painful treatment periods or, in some cases, reduction or cessation of all treatments due to kidney damage and/or bone pain serious adverse effects. The side effects with filgrastim or PEG-filgrastim are associated with dosage levels. Therefore, newer versions of GCSF (preferably PEGx-GCSF with improved PK profiles) are warranted to provide a clinically beneficial increase of neutrophils with reduced side effects. A drug formulation that can yield neutrophil increases similar to current treatments, but at lower dosages, therefore would be a desirable approach to improving conditions associated with neutropenia.

An additional, potential consequence of long-term GCSF therapy is the increased chance of developing a malignancy. Patients with severe chronic neutropenia (SCN), who require life-long GCSF therapy, are at an increased risk for myelodysplastic syndrome that is directly proportional to the time they have been treated with GCSF. It also is known that GCSF may exacerbate myelogenous cancers. Therefore, Neupogen is not recommended in patients with, e.g., myelodysplastic syndrome, chronic myelogenous leukemia, and secondary Acute Myeloid Leukemia (AML). Accordingly, it also would be advantageous to provide a GCSF therapy having proliferative activity that is more selective for normal cells, and therefore avoids or reduces the proliferation of cancer cells, as compared with currently existing treatments.

### SUMMARY OF THE INVENTION

Embodiments of the invention are directed to PEGx-GCSF, wherein x represents the number of PEG per GCSF and is an integer ranging from 4 to 8.

In embodiments of PEGx-GCSF, the PEG moiety has an average molecular weight from about 3 to about 15 kDa, or preferably from about 5 to about 6 kDa.

In certain embodiments of the inventive PEGx-GCSF, PEG is attached to GCSF through an amine originating from GCSF. In alternative embodiments, the PEGx-GCSF comprises a non-hydrolyzable linkage, for example, a urethane linkage.

In additional embodiments of the inventive PEGx-GCSF, GCSF is a protein having an amino acid sequence selected from the group consisting of SEQ ID NO: 1, SEQ ID NO: 3, SEQ ID NO: 4, and functional derivatives and homologs thereof. In further embodiments, the GCSF amino acid sequence is SEQ ID NO: 1 and each PEG is attached to a GCSF position selected from the group consisting of: the N-terminus, a lysine residue at position 17, a lysine residue at position 35, a lysine residue at position 41, a histidine residue at position 44, a histidine residue at position 53, a histidine residue at position 80, a histidine residue at position 157 and a histidine residue at position 171.

Embodiments of the invention are also directed to PEG[x]-GCSF, a composition that comprises a population of PEGx-GCSF, wherein [x] is the average value of x for the population, and wherein [x] is greater than or equal to about 4; wherein [x] is from about 4 to about 8; wherein [x] is from about 4 to about 6; or wherein [x] is from about 5 to about 6.

In certain embodiments, PEG[x]-GCSF is characterized by one or more of the following: PEG[x]-GCSF comprises less than 10% PEGx-GCSF wherein x is from 1 to 3; PEG[x]-GCSF comprises at least about 15% PEGx-GCSF wherein x is 4; PEG[x]-GCSF comprises at least about 30% PEGx-GCSF wherein x is 5; PEG[x]-GCSF comprises at least about 10% PEGx-GCSF wherein x is 6; and PEG[x]-GCSF comprises less than 15% PEGx-GCSF wherein x is 7.

In additional embodiments, PEG[x]-GCSF comprises at least about 15% PEGx-GCSF wherein x is in the range from 6 to 7; or comprises at least about 35% PEGx-GCSF wherein x is in the range from 5 to 7.

Additional embodiments are directed to a pharmaceutical formulation comprising a pharmaceutically active amount of PEGx-GCSF or PEG[x]-GCSF and a protein-free carrier.

Additional embodiments of the invention are directed to a method for preparing inventive PEGx-GCSF, wherein x is from 4 to 8, or PEG[x]-GCSF, wherein [x] is 4 or greater, the method comprising the steps of: (a) obtaining a GCSF solution having a concentration of at least about 5.0 mg/ml; (b) combining the GCSF solution with PEG, wherein the molar amount of PEG is about 65 to about 75 times the molar amount of the GCSF; (c) allowing sufficient time for the GCSF and PEG to react to produce PEGx-GCSF; (d) adding hydroxylamine in an amount sufficient to react with residual PEG; and (e) isolating PEG[x]-GCSF from unreacted PEG, N-hydroxysuccinimide and hydroxylamine. Individual PEGx-GCSF are further isolated from the population by methods known in the art for isolating purified protein conjugates, including methods of separating according to molecular weight.

The compositions of the invention provide unexpected utility in the treatment of various medical conditions where existing, commercially available GCSF and/or PEG-GCSF treatments may be contraindicated due to the occurrence of bone pain or the risk of cancer cell proliferation. Such medical conditions include severe congenital/chronic neutropenia, autoimmune/idiopathic neutropenias, as well as neutropenias associated with the treatment of cancers.

Additional advantages of the present invention will be readily apparent to those skilled in this art from the following detailed description, wherein only certain embodiments of the invention are shown and described. As will be realized, the invention is capable of other and different embodiments, and its several details are capable of routine modifications in various respects, all without departing from the invention. The present invention may be practiced without some or all of these specific details. Accordingly, the description is to be regarded as illustrative in nature, and not as restrictive.

### BRIEF DESCRIPTION OF THE FIGURES

Example embodiments of the disclosure may be understood by referring, in part, to the present disclosure and the accompanying drawings, which are briefly described below.

FIG. 1 represents the amino acid sequence of the predominant, fully processed human granulocyte colony stimulating factor (“GCSF”) (SEQ ID NO: 1). The corresponding DNA sequence is provided as SEQ ID NO: 2.

FIG. 2 describes the sequencing data for GCSF proteins used in certain embodiments of the invention described herein. FIG. 2 describes SEQ ID NO: 3 and SEQ ID NO: 4, respectively.

FIG. 3 provides a flow chart of a process for preparing the inventive PEGx-GCSF and PEG[x]-GCSF.

FIG. 4 is a representative bioanalyzer electropherogram obtained from analysis of inventive PEG[x]-GCSF samples.

FIG. 5 is a representative of SDS-PAGE analysis results for inventive PEG[x]-GCSF samples.

FIG. 6 is a potency graph illustrating results from a bioassay of inventive PEG[x]-GCSF (ANF-Rho) compared to a commercial PEG-GCSF (NEULASTA®, NLSTA). M-NFS-60 cells were treated with indicated GCSF compounds for 48 hours prior to viability staining. Data were normalized to untreated controls and fit to a three parameter logistic curve-fit model. Data represents mean and standard error of duplicate wells.

FIG. 7 is a quadrant gating of bivariate plots of fluorescent intensity of CD66 and CD14 cells, showing the effect of inventive PEG[x]-GCSF (ANF-Rho) vs. NEULASTA® on human CD34(+) cells in vitro. Cells were treated with 50 ng per ml of either the inventive PEG[x]-GCSF (ANF-Rho) or NEULASTA® for 14 days prior to surface staining for CD66 and CD14. Antigen expression was quantified by flow cytometry. Cell events appearing in the E4 gate are indicative of the granulocyte population.

FIG. 8A-8B are graphs of single-dose pharmacokinetics of various commercial PEG-GCSF (NEULASTA (ID) and inventive PEG[x]-GCSF samples in neutropenic rats. Rats were made neutropenic by injection of cyclophosphamide (CPA) on Day -1. Four different concentrations of inventive PEG[x]-GCSF (ANF-Rho) and NEULASTA® were administered subcutaneously at the indicated dosages on Day 1. Blood samples were obtained from the rats on the days indicated, and GCSF plasma concentrations were determined by ELISA. Data are mean and standard error for 8 rats per group. FIG. 8A is a linear plot, and FIG. 8B is a log plot of GCSF concentration.

FIG. 9A-9B are graphs showing the plasma exposure effects of commercial PEG-GCSF (NEULASTA®) and inventive PEG[x]-GCSF samples (lots 1-3) in neutropenic rats. FIG. 9A shows area under the curve (AUC) for three individual lots of inventive PEG[x]-GCSF at three different concentrations (100, 50 and 25 microgram per kilogram, i.e., μg/kg) and a single concentration of NEULASTA® (100 μg/kg). Asterisk indicates significant difference in inventive PEG[x]-GCSF AUC at indicated dosages compared to NEULASTA® administered at 100 microgram per kilogram (μg/kg). FIG. 9B shows linearity of PEG-GCSF plasma exposure and dosage. AUC values for Lot 1, Lot 2, and Lot 3 of inventive PEG[x]-GCSF were pooled and subjected to linear regression analysis. Pooled mean and 95% confidence intervals are shown above each data set. Dotted line and shaded area indicates mean AUC and upper and lower 95% confidence intervals of 100 μg/kg NEULASTA® treatment group. Asterisks indicate significant differences (p<0.05) by ANOVA and Dunnet's multicomparison tests post-hoc analysis as compared to NEULASTA® treated group.

FIG. 10 is a graph showing representative changes in neutrophil cell counts in neutropenic rats treated daily with inventive PEG[x]-GCSF lot 1, NEULASTA® or formulation buffer (FB). Rats were made neutropenic by injection of cyclophosphamide (CPA) on Day −1. On Day 1 and after, rats received daily injections of PEG[x]-GCSF (100 μg/kg), NEULASTA (100 μg/kg) or vehicle solution (FB). Blood samples were obtained from the rats on the days indicated to determine absolute neutrophil counts (ANC). Data are means and standard error for 8 rats per group. Shaded area indicates ANC values associated with initial neutrophil release, which were not included in area under the curve calculations.

FIG. 11A-11B illustrates the absolute neutrophil counts (ANC) from PEG[x]-GCSF and NEULASTA® dosed neutropenic rats. FIG. 11A shows ANC values plotted as a function of hours post administration to determine AUC, using the second rise ANC peak shown in FIG. 10. Values for each PEG[x]-GCSF lot at each dose were pooled, and data prior to 96 hours (representing release of pre-formed neutrophils) was excluded from analysis. Asterisks above and below data set represent significant difference (p<0.05) by ANOVA of pooled dosages of PEGx-GCSF compared to formulation buffer and NEULASTA® treatment groups, respectively. FIG. 11B shows data grouped of the three concentrations from three separate lots of the PEG[x]-GCSF at 25 μg/kg (squares), 50 μg/kg (inverted triangles) and 100 μg/kg (circles). The shaded area represents the values for 100 μg/kg NEULASTA® and 95% CI (confidence interval). Correlation analysis between ANC-AUC and Plasma AUC of all lots with an r2 value of 0.64 indicates significant correlation between drug levels and ANC pharmacodynamics.

### DETAILED DESCRIPTION

### Definitions

“Substantially homologous,” in reference to an amino acid sequence, is defined herein as a sequence with at least 70%, typically at least about 80%, and more typically at least about 90% identity to another amino acid sequence, as determined by the FASTA search method in accordance with Pearson and Lipman, Proc. Natl. Acad. Sci. USA 85, 2444-2448 (1988).

As used herein, the term “N-terminus,” “amino-terminus,” or analogous terms when used in the context of a covalent linkage of a protein to another molecule refer to a covalent linkage via the amino-terminal a-amino group of the protein.

As used herein, the term “wild type” or “native” refers to a protein or polypeptide in its operative or functional form, typically as it is found naturally functioning in the body. These terms also refer to the protein in a form in which it has not been artificially modified or altered. The terms can thus relate to recombinant proteins. Accordingly, the terms can refer to a protein with an altered glycosylation pattern, including lack of glycosylation, relative to that as produced in the animal from which the nucleic acid and/or amino acid sequence of the protein was originally derived.

The term “ANF-Rho” is used herein to refer to an exemplary sample of PEG[x]-GCSF of the present invention used in the Examples. See, e.g., Example 3 and Table 2.

NEULASTA® is the brand name of PEGfilgrastim, a PEGylated form of the recombinant human granulocyte colony-stimulating factor (GCSF) analog filgrastim. The drug is prepared by coupling a 20 kDa polyethylene glycol (PEG) molecule to the N-terminus of the filgrastim protein.

### GCSF

In general, a GCSF protein useful in the practice of this invention may be of any form isolated from mammalian organisms, a product of prokaryotic or eukaryotic host expression of exogenous DNA sequences obtained by genomic or cDNA cloning or by DNA synthesis or alternatively a product of chemical synthetic procedures or by endogenous gene activation. Thus, the protein can be of a natural or recombinant source obtained from tissue, mammalian/microbial cell cultures, plant cell cultures, transgenic animals, yeasts, fungi and/or transgenic plants. Suitable prokaryotic hosts include various bacteria such as E. coli; suitable eukaryotic hosts include yeasts such as S. cerevisiae or Pichia pastoris, mammalian cells such as Chinese hamster ovary cells or monkey cells, transgenic animals such as mice, rabbit, goat, sheep, insect or plant cell culture and transgenic plants such as Physcomitrellapatens (a moss). Depending upon the host employed, the protein expression product may be glycosylated with mammalian, plant or other eukaryotic carbohydrates, or it may be non-glycosylated.

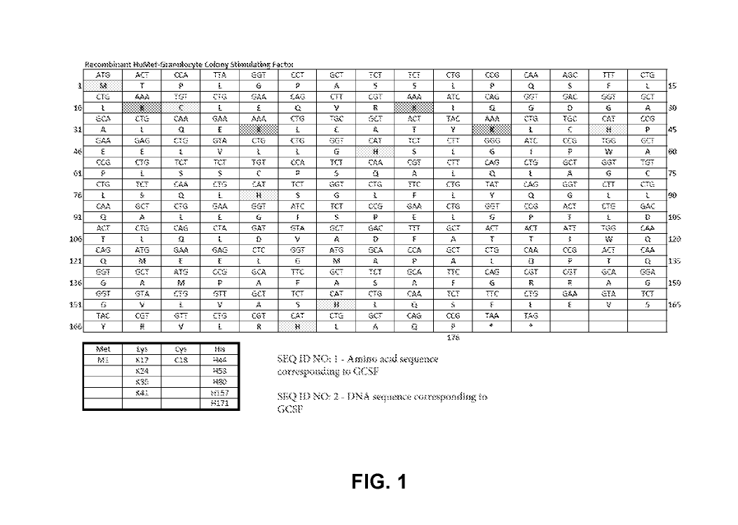

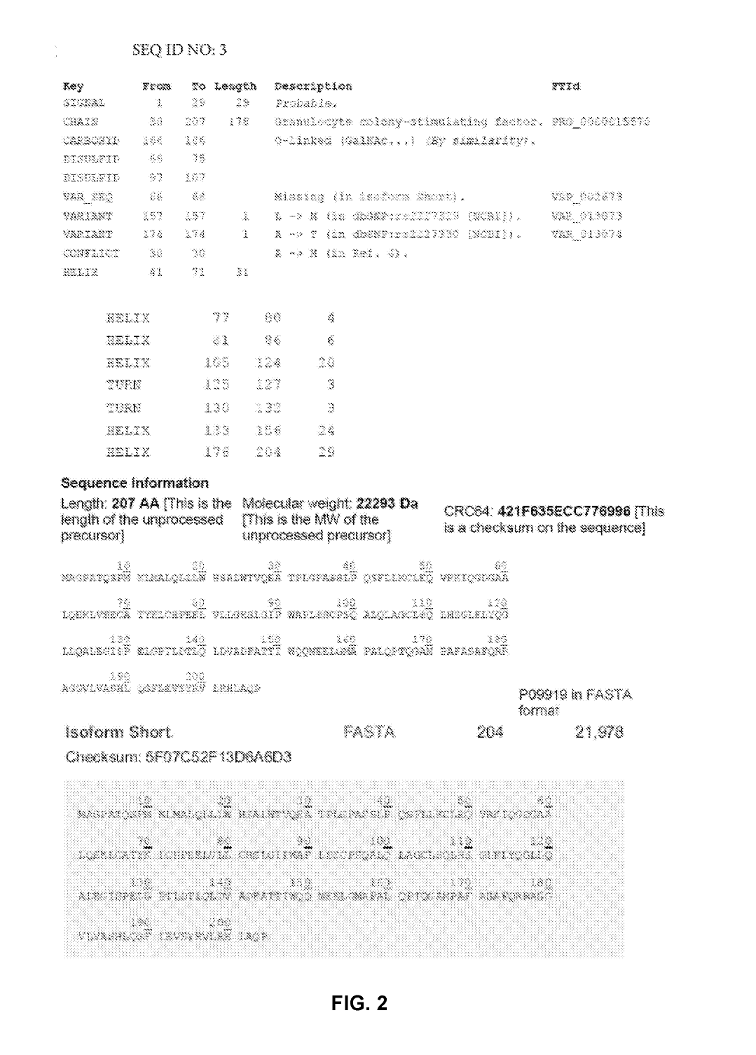

As used herein, the term “GCSF” or granulocyte colony stimulating factor includes a protein having the amino acid sequence set out in SEQ ID NO: 1 (FIG. 1) or an amino acid sequence substantially homologous thereto, whose biological properties relate to the stimulation of white blood cell production. As used herein, the term GCSF includes such proteins modified deliberately, as for example, by site directed mutagenesis, or accidentally through mutations; such that they have additions, deletions, or substitutions of amino acid residues with respect to native GCSF. These terms include both natural and recombinantly produced human GCSF. GCSF refers to both the naturally occurring or recombinant protein, typically human, as obtained from any conventional source such as tissues, protein synthesis, cell culture with natural or recombinant cells.

A GCSF expression product useful in the practice of the invention may also include an initial methionine amino acid residue at position 1. The present invention contemplates the use of any and all such forms of GCSF, although recombinant GCSF, especially E. coli-derived, is typical. Certain GCSF analogues have been reported to be biologically functional, and these may also be conjugated according to the present invention. These GCSF analogues may include those having amino acid additions, deletions and/or substitutions as compared to the GCSF amino acid sequence according to SEQ ID NO: 1. In certain embodiments, the sequence includes an insertion of amino acids as compared to SEQ ID NO: 1, such as, for example, an insertion of VSE at positions 36, 37 and 38 of SEQ ID NO: 1. In certain embodiments, the sequence is as in SEQ ID NO: 3 or SEQ ID NO: 4.

The term “GCSF” as used herein encompasses proteins having the activity of GCSF described above, including the natural human glycoprotein GCSF, mutants of GCSF, glycosylated GCSF, non-glycosylated GCSF and/or otherwise modified structural and/or functional variants of GCSF. In a further embodiment, GCSF has the amino acid sequence identified in SEQ ID NO: 1 that corresponds to recombinant GCSF produced in bacteria, having 174 amino acids and an extra N-terminal methionyl residue. Amino acid sequences of biologically active GCSF, which differ from SEQ ID NO: 1 in that they do not contain a methionyl residue at position 1, are also included.

### PEG

The term “PEG” generally refers to a polyalkylene glycol compound or derivative thereof, with or without linkers or activating moieties. The term PEG as used herein includes, but is not limited to, polyethylene glycol homopolymers, copolymers of ethylene glycol with propylene glycol and derivatives and equivalents thereof, wherein said homopolymers and copolymers are unsubstituted or substituted, for example, at one end with an alkyl group. The PEG polymers for use with the present invention can be linear, branched, comb or star-shaped with a wide range of molecular weights. The average molecular weight of the PEG for use with embodiments of the present invention can range from 5 to about 100 kDa.

Numerous derivatives of PEG and methods for making them and conjugating them to a protein are known in the art and are suitable for use in the present invention. One particularly preferred PEG for use in the invention is a PEG having one end of the polymer terminating with a relatively inert group, such as a lower C₁₋₆ alkoxy group. Preferably, the PEG is a monomethoxy-PEG (commonly referred to as mPEG), which is a linear form of PEG wherein one terminus of the polymer is a methoxy (—OCH₃) group.

Even more preferably, the PEG used in the invention is an “activated mPEG” in which one end of the linear PEG terminates with a methoxy group and the other end terminates with a linker appropriate for coupling to the preferred sites on GCSF in order to facilitate PEGylation with a desired activated mPEG

Preferred linkers include amine reactive linkers, i.e., synthetic chemical groups that will form chemical bonds with primary amines. These include isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters. Most of these amine reactive linkers conjugate to amines by either acylation or alkylation.

Exemplary linkages are hydrolytically stable, and water soluble. Representative suitable linkers can comprise any combination of amide, a urethane (also known as carbamate), amine, thioether (also known as sulfide), or urea (also known as carbamide) groups.

In a particular embodiment, methoxylated PEG (“mPEG”) can be activated for subsequent covalent attachment to amino groups by methods well known in the art, i.e., mPEG can be modified to contain varying reactive moieties suitable for subsequent attachment to proteins via amino acid residues containing available amino residues, e.g., lysinyl residues. Such activated PEGs include mPEG-succinimidyl succinate (“SS-PEG”), mPEG-succinimidyl carbonate (“SC-PEG”), mPEGimidate, and mPEG-cyanuric chloride. In a preferred embodiment, the linkers are selected to provide PEG-GCSF linkages that are stable to hydrolysis.

In certain embodiments, the average molecular weight of the PEG for use with the present invention is in the range from about 2 to about 50 kDa, about 3 to about 25 kDa, about 4 to about 10 kDa or any subrange defined by two of the endpoints provided herein, including any single number integer (whole number) or non-integer (fraction) found within these ranges such as 4.5, 5, 5.6 and 6 kDa.

In a particular embodiment, the PEG for use in the various embodiments of the present invention has an average molecular weight of about 5 to 6 kDa SC-PEG; more particularly about 5.6 kDa SC-PEG The PEGx-GCSF resulting from reaction of GCSF primary amino groups with SC-PEG comprises urethane linkages that are stable to hydrolysis, unlike the hydrolytically labile linkages in the multi-PEGylated conjugates of the Tanaka et al. and Satake-Ishikawa et al., discussed above.

### PEGx-GCSF

One embodiment of the present invention is directed to “PEGx-GCSF” which, as defined herein, is a GCSF conjugate comprising x number of PEG moieties covalently attached thereto, wherein x is an integer from 4 to 7. Particular embodiments of PEGx-GCSF include wherein x is 4, 5, 6, 7 and 8.

In certain embodiments, each PEG is attached to GCSF through an amine moiety originating from GCSF, for example, the N terminus, or any lysine or histidine residue. In these particular embodiments, covalent attachment is formed by reaction between PEG activated with an amino-reactive linker and a GCSF amine moiety. In particular embodiments, upon reaction with an amine, the amino-reactive linker forms a non-hydrolysable linkage to GCSF. In further embodiments, PEGx-GCSF comprises a non-hydrolysable linkage, for example, a urethane linkage.

Embodiments of PEGx-GCSF include wherein GCSF is a protein having an amino acid sequence selected from the group consisting of SEQ ID NO: 1, SEQ ID NO: 3, SEQ ID NO: 4, and functional derivatives and homologs of any of these sequences. In particular embodiments, the amino acid sequence is SEQ ID NO: 1, a functional derivative of SEQ ID NO: 1, or a homolog of SEQ ID NO: 1, where the GCSF has a lysine residue at position 17, a lysine residue at position 35, a lysine residue at position 41, and optionally, a histidine residue at position 44, a histidine residue at position 53, a histidine residue at position 80, a histidine residue at position 157 and a histidine residue at position 171. In related embodiments of PEGx-GCSF, each PEG is attached to GCSF at a position selected from the group consisting of: the N-terminus, the lysine residue at position 17, the lysine residue at position 35, the lysine residue at position 41, and optionally at the histidine residue at position 44, the histidine residue at position 53, the histidine residue at position 80, the histidine residue at position 157 and the histidine residue at position 171.

Specific embodiments are directed to PEGx-GCSF, where GCSF is a protein having the amino acid sequence of SEQ ID NO: 1, and wherein each PEG is attached to a GCSF originating amine, such as the N-terminus, a lysine, or a histidine residue. For example, embodiments where PEG is attached to a GCSF position selected from the group consisting of: the N-terminus, a lysine residue at position 17, a lysine residue at position 35, and a lysine residue at position 41. In a further embodiment, PEG is attached to a GCSF position selected from the group consisting of: a histidine residue at position 44, a histidine residue at position 53, a histidine residue at position 80, a histidine residue at position 157 and a histidine residue at position 171.

In certain embodiments of PEGx-GCSF, the average molecular weight of PEG is in the range from about 2 to about 50 kDa, about 3 to about 25 kDa, about 4 to about 10 kDa or any sub-range defined by two of the endpoints provided herein, including any single number integer (whole number) or non-integer (fraction) found within these ranges such as 4.5, 5, 5.6 and 6 kDa. Particular embodiments of PEGx-GCSF include those wherein PEG has an average molecular weight from about 5 to about 6 kDa, preferably 5.6 kDa.

### PEG[x]-GCSF

An embodiment of the invention is directed to “PEG[x]-GCSF” which, as defined herein, is a composition that comprises a population, including various proportions of any individual PEGx-GCSF described above, wherein [x] is the average value of x for the population, and wherein [x] is a positive number (including fractional values) greater than or equal to about 4, for example from about 4 to about 8; from about 4 to about 6; or from about 5 to about 6. PEG[x]-GCSF encompasses embodiments comprising a “heterogeneous population” wherein the PEGx-GCSF conjugates have different values of x, wherein PEG is attached at different sites on GCSF molecules, and/or wherein the PEG has different molecular weights.

Embodiments of PEG[x]-GCSF include populations of any of the various individual PEGx-GCSFs described herein, including PEGx-GCSF wherein the average molecular weight of PEG is in the range from about 2 to about 50 kDa, about 3 to about 25 kDa, about 4 to about 10 kDa. Particular embodiments of PEGx-GCSF include those wherein PEG has an average molecular weight from about 5 to about 6 kDa, preferably 5.6 kDa.

Certain embodiments of PEG[x]-GCSF are characterized by one or more of the following: comprising less than 10%, less than 8% or less than 5% PEGx-GCSF wherein x is from 1 to 3; comprising at least about 15%, at least about 18%, at least about 20%, at least about 25%, or at least about 30% PEGx-GCSF wherein x is 4; comprising at least about 30%, at least about 35%, or at least about 40% PEGx-GCSF wherein x is 5; comprising at least about 10%, at least about 12%, or at least about 15% PEGx-GCSF wherein x is 6; comprising at least about 3%, at least about 5%, and/or less than about 15% PEGx-GCSF wherein x is 7; comprising at least about 15%, at least about 20%, at least about 25%, or at least about 35% PEGx-GCSF wherein x is in the range from 6 to 7; and comprising at least about 35%, at least about 40%, at least about 45%, at least about 50%, at least about 60%, at least about 75% or at least about 80% PEGx-GCSF wherein x is in the range from 5 to 7. Additional embodiments of PEG[x]-GCSF may include those comprising less than about 15% , less than about 12%, or less than about 10% PEGx-GCSF wherein x is 7.

Embodiments of PEG[x]-GCSF include populations of any of the various individual PEGx-GCSFs described herein, wherein [x], the average value of the x for the population, is greater than 4. For example, embodiments of PEG[x]-GCSF include compositions of PEGx-GCSF where PEG is attached to GCSF through an amine originating from GCSF, such as the N-terminus, a lysine, or a histidine; wherein PEGx-GCSF comprises a non-hydrolyzable linkage, for example a urethane linkage; wherein GCSF is a protein having an amino acid sequence of SEQ ID NO: 1, SEQ ID NO: 3, SEQ ID NO: 4, and functional derivatives and homologs of any one of these sequences; wherein each PEG is attached to GCSF according to SEQ ID NO: 1 or a derivative or homologue thereof, at a position selected from the group consisting of: the N-terminus, the lysine residue at position 17, the lysine residue at position 35, the lysine residue at position 41, the histidine residue at position 44, the histidine residue at position 53, the histidine residue at position 80, the histidine residue at position 157 and the histidine residue at position 171.

In some embodiments, PEG[x]-GCSF comprises a population of PEGx-GCSF characterized by one or more of the following:
from about 0% to about 5% of PEGx-GCSF wherein x is 3;
from about 22% to about 32% of PEGx-GCSF wherein x is 4;
from about 38% to about 42% of PEGx-GCSF wherein x is 5;
from about 18% to about 28% of PEGx-GCSF wherein x is 6; and
from about 0% to about 9% of PEGx-GCSF wherein x is 7.

Another embodiment of PEG[x]-GCSF comprises a population of PEGx-GCSF wherein the PEG is attached to the GCSF through a urethane linkage and, optionally, wherein the PEG has an average molecular weight molecular weight from about 3 to about 15 kDa, more preferably, from about 5 to about 6 kDa. In a particular embodiment, the PEG[x]-GCSF consists of a population of PEGx-GCSF wherein the PEG is attached to the GCSF through a urethane linkage and, optionally, wherein the PEG has an average molecular weight molecular weight from about 3 to about 15 kDa, more preferably, from about 5 to about 6 kDa.

### Process of Conjugation

The following process is followed in order to produce the PEGx-GCSF conjugates where x, i.e., the number of PEG per GCSF, is from 4 to 8, and the PEG[x]-GCSF conjugate populations where [x], i.e., the average number of PEG per GCSF present in the population of PEGx-GCSF, is 4 or greater in accordance with the present invention.

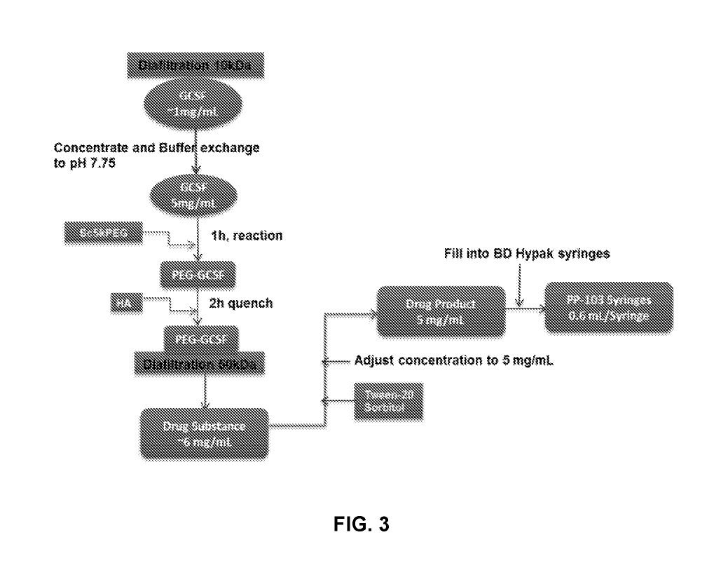

As shown in FIG. 3, GCSF protein is concentrated to about 5.0 mg/ml and subject to buffer exchange using a 10 kDa diafiltration membrane. The reaction vessel, equipped with a stirring mechanism, is filled with the protein solution. A dual-blade impeller system set to the desired blade depth, is submerged into the vessel and turned on. SC-PEG powder (5 kDa), at a molar excess of from about 65 to about 75 times the amount of protein, is slowly added to the reaction vessel over a period of about 15 minutes. The pH is monitored and maintained at about 7.75 while the reaction continues about an additional 45 minutes. Following this, hydroxylamine (HA) is added to the reaction vessel and mixed for about another 2 hours to quench residual reactive PEG and to strip weakly associated PEGs from the product. Throughout the reaction process, the contents of the reaction vessel are maintained at ambient temperature (i.e., “room temperature”). The reaction mixture is diafiltered two times using a 50 kDa membrane to remove the residual (i.e., unreacted) PEG, N-hydroxysuccinimide and hydroxylamine. The obtained “drug substance” is concentrated to between 5.0 and 6.0 mg/ml. The “drug product” is then formulated through the addition of TWEEN 20® (polyethylene glycol sorbitan monolaurate, Sigma-Aldrich, St. Louis, Mo.) and sorbitol, adjusting volume to a final drug product concentration from about 2 to about 10 mg/ml, preferably about 5.0 mg/ml. The drug product is dispensed into sterile container closures such as vials or syringes.

### Pharmaceutical Formulations

In certain embodiments, the invention relates to a pharmaceutical formulation comprising a PEGx-GCSF conjugate, wherein x is from 4 to 8, or a PEG[x]-GCSF population of individual conjugates, wherein [x] is 4 or greater, as described herein, optionally in a pharmaceutically acceptable carrier. In certain embodiments, the carrier is substantially protein free.

The formulations of the invention may be further rendered suitable for injection by mixture or combination with an additional pharmaceutically acceptable carrier or vehicle by methods known in the art. Among the pharmaceutically acceptable carriers for formulating the products of the invention are saline, human serum album, human plasma proteins, etc. The invention also relates to pharmaceutical compositions comprising a conjugate as described above and a pharmaceutically acceptable excipient and/or carrier. Such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like.

Pharmaceutical compositions of the invention comprise effective amounts of PEGx-GCSF conjugate, wherein x is from 4 to 8, or a PEG[x]-GCSF population of individual conjugates, wherein [x] is 4 or greater, of the present invention together with pharmaceutically acceptable diluents, preservatives, solubilizers, emulsifiers, adjuvants and/or carriers. Such compositions includes diluents of various buffer content, such as Tris-HCl, acetate, phosphate, pH and ionic strength; additives such as detergents and solubilizing agents such as TWEEN 80® (non-ionic oleic acid, ≧58.0% (balance primarily linoleic, palmitic, and stearic acids) average mol wt 1310, available from Sigma Aldrich—also referred to as Polysorbate 80), antioxidants such as ascorbic acid and sodium metabisulfite, preservatives such as benzyl alcohol and bulking substances such as lactose or mannitol; incorporation of the material into particulate preparations of polymeric compounds such as polylactic acid, polyglycolic acid, etc. or into liposomes. Such compositions may influence the physical state, stability, rate of in vivo release and rate of in vivo clearance of the PEGx-GCSF conjugates according to the present invention.

PEGx-GCSF conjugates, wherein x is from 4 to 8, or PEG[x]-GCSF populations of individual conjugates, wherein [x] is 4 or greater, prepared in accordance with this invention may be formulated in pharmaceutical compositions suitable for injection with a pharmaceutically acceptable carrier or vehicle by methods known in the art. See, e.g., WO97/09996, WO97/40850, WO98/58660, and WO99/07401, the entire contents of which are incorporated herein by reference. The compounds of the present invention may be formulated, for example, in 10 mM sodium/potassium phosphate buffer at pH 7 containing a tonicity agent, e.g. 132 mM sodium chloride. Optionally, the pharmaceutical composition may contain a preservative.

The pharmaceutical compositions generally comprise PEGx-GCSF conjugates, wherein x is from 4 to 8, or PEG[x]-GCSF populations of individual conjugates, wherein [x] is 4 or greater, prepared in accordance with this invention, a multiply charged inorganic anion in a pharmaceutically acceptable buffer suitable to keep the solution pH in the range of from about 4.0 to about 7.0 (but most preferably at the lower end of this range; i.e., about 4.0), and optionally one or more pharmaceutically acceptable carriers and/or excipients.

### Methods of Use

In another aspect of the invention, a method is provided for increasing white blood cell count in a patient in need thereof, comprising administering to said patient a pharmaceutical formulation of the invention. In certain embodiments, the patient is at risk of, or suffering from, neutropenia. In certain other embodiments, the patient is being treated with an agent that decreases his/her white blood cell count. In certain embodiments, the patient has decreased endogenous levels of GCSF. In certain other embodiments, the patient is undergoing radiation treatment. The patient may be suffering from lung cancer, lymphoma, breast cancer, bone marrow transplantation, testicular cancer, AIDS-related malignancies, myelodysplastic disorders, acute leukemia, congenital and cyclic neutropenias or aplastic anemia (see Mortsyn, et al.(1998) Filgrastim (r-metHuGCSF). In Clinical Practice, 2^{nd} Ed., Marcel Dekker, Inc., New York, N.Y.). In certain embodiments, the formulation is administered to a patient at risk of infection.

The following are examples of primary neutropenias, due to intrinsic defects in myeloid cells or their precursors, for which the inventive method of treatment is expected to be useful: Aplastic anemia; Chronic idiopathic neutropenia, including benign neutropenia; Cyclic neutropenia; Myelodysplasia; Neutropenia associated with dysgammaglobulinemia; Paroxysmal nocturnal hemoglobinuria; Severe congenital neutropenia (Kostmann syndrome); and Syndrome-associated neutropenias (e.g., cartilage-hair hypoplasia syndrome, Chédiak-Higashi syndrome, dyskeratosis congenita, glycogen storage disease type IB, Shwachman-Diamond syndrome, Myelokathexis syndrome, congenital immunologic deficiency syndromes).

The following are exemplary causes of secondary or acquired neutropenias for which the inventive method of treatment is expected to be useful: Alcoholism; Autoimmune neutropenia, including chronic secondary neutropenia in AIDS; Autoimmune diseases (e.g., Felty's syndrome/Rheumatoid arthritis, Sjogren's syndrome, Systemic lupus erythematosus); Bone marrow replacement or stem cell transplantation; Cancer (e.g., bone marrow infiltration by leukemia, myeloma, lymphoma, or metastatic solid tumors—e.g., breast, prostate cancers); Tγ lymphoproliferative disease; Febrile neutropenias caused by cytotoxic chemotherapy or radiation therapy; Drug-induced neutropenia; Folate or vitamin B₁₂ deficiency (megaloblastic anemias); Hemodialysis; Hypersplenism; Infection (e.g., parvovirus, hepatitis viruses, malaria, Lyme disease, salmonella, sepsis); Myelofibrosis (i.e. granulomatous infections); Gaucher's disease; Poisoning (e.g., Arsenic); and Primary immunodeficiencies—e.g., X-linked, Common Variable Immune Deficiency (CVID), X-linked Agammaglobulinemia (XLA), WHIM syndrome, Wiskott-Aldrich Syndrome and GATA2 deficiency.

The pharmaceutical compositions of the invention may be especially useful in the treatment of certain myeloid cancers (e.g., Acute myeloid leukemia, Chronic myelogenous leukemia, Acute promyelocytic leukemia) for which the administration of currently commercially available GCSF products is contraindicated. This is due to the unexpected selectivity of the inventive PEGx-GCSF, and inventive PEG[x]-GCSF populations thereof, in causing the proliferation of normal white blood cells, while avoiding or reducing the proliferation of cancer cells, as demonstrated in Example 3, below.

Moreover, in addition to the especially advantageous treatment of cancer patients provided by the present invention, there are patients with conditions that can result in severe chronic neutropenia (SCN) that require life-long GCSF therapy. These patients are at an increased risk for myelodysplastic syndrome that is directly proportional to their cumulative exposure to GCSF protein. Thus, the inventive compositions, which avoid or reduce the proliferation of cancer cells, also could provide a unique benefit to these patients.

Further, in addition to treating neutropenia, GCSF has been used in peripheral blood stem cell mobilization in autologous transplant patients and in allogeneic donors. Prior to a transplant, the donor or patient is treated with GCSF to increase the number of progenitor stem cells, so there is a better harvest of stem cells and therefore better likelihood of success of the transplant procedure. The pharmaceutical compositions of the invention are expected to be particularly useful for this purpose, in view of their enhanced bioactivity, as demonstrated in Example 4, below.

The PEGx-GCSF conjugates, wherein x is from 4 to 8, or PEG[x]-GCSF populations of individual conjugates, wherein [x] is 4 or greater, prepared in accordance with this invention also have utility in the treatment of severe sepsis and septic shock due to down-regulation of the GCSF receptor by, e.g., endotoxin.

In certain embodiments, an inventive formulation is provided in a single dose during a course of chemotherapy. In some embodiments, the formulation is provided as multiple doses over the course of chemotherapy. In certain embodiments, the formulation is administered once daily, once weekly, once every two weeks or once a month. The formulation can be administered within twenty-four hours of a dose of chemotherapy. In certain embodiments, the formulation is administered at least 14 days before a dose of chemotherapy. However, as explained in greater detail below, the inventive formulations provide much greater dosing flexibility than is the case with the commercially available NEULASTA® product. The inventive formulations advantageously may be administered to a patient at any time during chemotherapy.

In certain embodiments, the formulation is administered as an injection. In some embodiments, the formulation is suitable for multiple administration routes including subcutaneous, intramuscular and intraperitoneal. In other embodiments, the formulation is suitable for intravenous administration. The formulation can also be provided as an orally available form. A patient may receive a dose at least about once a week. In other embodiments, the patient receives a dose at least about once every two weeks, at least about once every three weeks, or at least about once every month.

The therapeutically effective amount is that amount of PEGx-GCSF conjugates, wherein x is from 4 to 8, or PEG[x]-GCSF populations of individual conjugates, wherein [x] is 4 or greater, prepared in accordance with this invention necessary for the in vivo biological activity of causing bone marrow cells to increase production of white blood cells. The exact amount of PEGx-GCSF or PEG[x]-GCSF is a matter of preference, subject to such factors as the exact type of condition being treated, the condition of the patient being treated, as well as other ingredients in the composition. The pharmaceutical formulations containing the PEGx-GCSF or PEG[x]-GCSF may be formulated at a strength effective for administration by various means to a human patient experiencing disorders characterized by low or defective white blood cell production. Average therapeutically effective amounts of the PEGx-GCSF or PEG[x]-GCSF may vary and in particular should be based upon the recommendations and prescription of a qualified physician. For example, 0.01 to 10 μg per kg body weight, typically 0.1 to 3 μg per kg body weight, may be administered, e.g., once a chemotherapy cycle. Alternatively, the pharmaceutical compositions of the invention may contain a fixed dose of the PEGx-GCSF or PEG[x]-GCSF , e.g., from 1 to 10 mg, or from 2-9 or about 6 mg in a fixed dose formulation useful for a host over 45 kg. However, as demonstrated in Example 4, below, these amounts may be decreased in view of the inventive compositions' dramatically increased level of in vivo activity as compared with, e.g., NEULASTA®.

### EXAMPLES

### Example 1

### Synthesis of the Inventive PEG[x]-GCSF

Example PEGx-GCSF conjugates, wherein x is from 4 to 8, or PEG[x]-GCSF populations of individual conjugates, wherein [x] is 4 or greater, were produced in accordance with the general procedure set forth in the “PROCESS OF CONJUGATION” section above, utilizing four primary steps: (1) Diafiltration pH 7.75-10 kDa, (2) PEGylation reaction, (3) Diafiltration pH 4.0-30 kDa/50 kDa and filtration, and (4) Fill finish into sterile borosilicate stoppered glass vials or BD Hypak syringes (Becton Dickinson, Franklin Lakes, N.J.), as presented in greater detail below.

### Diafiltration pH 7.75-10 kDa

GCSF protein was buffer exchanged into PEGylation buffer (100 mM phosphate buffer at pH 7.75) using 10 kDa membrane with 20 volumes of PEGylation buffer. After buffer exchange, the solution was concentrated to 5 mg/ml solution as determined by UV spectrophotometry.

### PEGylation Reaction and Addition of Hydroxylamine

The concentrated GCSF (5 mg/ml) in phosphate buffer in a 600 mL glass beaker was slowly stirred as PEG was added under the following conditions: pH of 7.75; ambient temperature; and 1-hour reaction time. SC-PEG powder (5 kDa), at a molar ratio (PEG:GCSF) of 65× to 75×, was added the slowly, and was under constant stirring at 150 rpm throughout the addition, which was completed in about 15 minutes. Reaction pH was monitored and maintained at 7.75 by addition of 10N NaOH during PEG addition as necessary; subsequently the pH remained constant. After stirring for 1 hour, the reaction was terminated by adding 1.2 M hydroxylamine hydrochloride (HA). Addition of hydroxylamine purges the labile PEG additions at histidines and improves product homogeneity and stability. A mixture of PEGylated GCSF protein was formed from these reactions, i.e., PEG[x]-GCSF, with PEG attached at various sites on the GCSF molecules.

### Diafiltration at pH 4.0-30 kDa and Filtration

The reaction mixture then was buffer exchanged to sodium acetate pH 4 buffer using a 30/50KDa diafiltration system with 20 volumes of acetate buffer (10 mM sodium acetate, pH 4.0). PEG[x]-GCSF was collected in the retentate; N-hydroxysuccinimide (NETS), hydroxylamine and 5 kDa free PEG were removed in permeate. A second identical diafiltration then was performed to promote further removal of process related impurities. After buffer exchange, the solution was concentrated to ˜5.5 mg/ml solution. The resulting PEG[x]-GCSF solution was filtered through a 0.2 μm filter aseptically and stored at 2-8° C. in sodium acetate buffer, as the final purified PEG[x]-GCSF (also referred to as the “Drug Substance”). The yield of the product obtained from the batches listed was estimated by measuring OD₂₈₀ using a spectrophotometer.

Fill/Finish into BD/Hypak Glass Syringes

Concentration of the obtained PEG[x]-GCSF was adjusted to 5 mg/ml using sodium acetate buffer at pH 4 after adding the formulation excipients at estimated volume at 5 mg/ml. The final “Drug Product” was then formulated through the addition of TWEEN 20® (polyethylene glycol sorbitan monolaurate, Sigma-Aldrich, St. Louis, Mo.) and sorbitol, adjusting volume to a final drug product concentration from about 2 to about 10 mg/ml, preferably about 5.0 mg/ml and filled into BD Hypak glass syringes at 0.6 ml/ syringe using a hand filling tool and repeater pipettor. Final drug product also has been formulated from to 2.0 and 5.0 mg/ml and aseptically dispensed into sterile glass vials with stoppers.

### Example 2

### Characterization of PEG[x]-GCSF

This Example describes analytical work that was performed in order to characterize the inventive embodiments of individual PEGx-GCSF and PEG[x]-GCSF populations exemplified herein, particularly with respect to the number x and location of PEG molecules attached to a GCSF protein. In addition, the attributes of inventive PEG[x]-GCSF were compared with those of the multi-PEGylated conjugates described in Tanaka et al. (1991) Cancer Research 51:3710-3714 and Satake-Ishikawa et al. (1992) Cell Structure and Function 17:157-160.

### Bioanalyzer Procedure:

The Agilent 2100 Bioanalyzer is a microchip-based capillary electrophoresis system which can rapidly separate proteins based on size, and provides automated dye-based visualization and quantification capabilities. 4 μl of a PEG-GCSF sample—either drug substance (i.e., the inventive PEG[x]-GCSF formulated in 10 mM sodium acetate, pH 4) or drug product (i.e., drug substance to which Sorbitol and TWEEN®-20 have been added)—was diluted to 1 mg/ml and combined with 2 μl of Agilent Denaturing Solution with Dithiothreitol (DTT) (7 μl of 1M solution added to new vial of Agilent Denaturing solution) and heated to 95-100° C. for 5 minutes. The denatured sample was diluted further by adding 84 μl of water prior to loading upon the chip. The Agilent Protein 230 kit ladder and an in-house-produced 5K PEG ladder were prepared identically to the PEG-GCSF sample. The 5K PEG ladder is a mixture of PEG-GCSF conjugates with extents of PEGylation ranging from 1 to 4 and is used as a check for system performance and as a reference for evaluation of the composition of the PEG-GCSF samples being analyzed. For each sample analyzed, an electropherogram is captured which represents the peak area for each separated protein species. For PEG-GCSF, typically 4-5 peaks are seen which correspond variously to GCSF containing 3-7 PEG's. A weighted average of the peak area of each PEGylated species will produce the average PEG number for the sample tested.

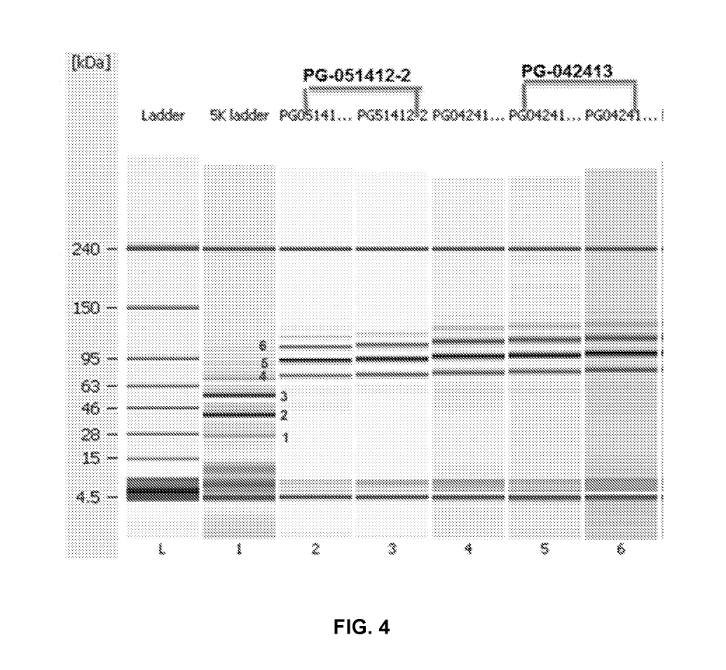

In the Bioanalyzer gel image shown in FIG. 4, the lane at the far left contains molecular weights for the proteins that are analyzed in the next lane labeled “Ladder.” The “5K Ladder” in the next column contains a PEG-GCSF sample comprised of various amounts of PEG1-GCSF, PEG2-GCSF, and PEG3-GCSF, with a small amount of PEG4-GCSF. PEG-GCSF lot PG-051412-2 is applied to the next two lanes, followed immediately by PEG-GCSF lot PG-042413, first as a “spacer” and then applied in duplicate for analytical purposes. The numbers at the border of lanes 2 and 3 represent the number x of PEG bound to GCSF for each PEGx-GCSF species displayed. The molecular weight of PEG used for all batches was 5.6 kDa.

It should be noted that the PEGx-GCSF species labeled as having 4 PEG's per GCSF molecule (i.e., where x=4) runs at an apparent molecular weight greater than 63 kDa, based on the migration of the 63 kDa protein marker, despite the fact that the true average molecular weight is approximately 41 kDa. As discussed below for the data reported in the Tanaka paper, the molecular weight of the PEGylated compounds is overestimated when using globular proteins as molecular weight markers. This is true whether analysis is by the Bioanalyzer, as described here, or SDS-PAGE, as described in the Tanaka paper.

The bands in FIG. 4 are quantifiable and are used for area % determination of each PEGx-GCSF species. Table 1, immediately below, contains a summary of the ranges of results obtained for a number of different batches of inventive PEG[x]-GCSF:

TABLE 1

| PEGx-GCSF | Percentage Composition |
|---|---|
| x = 3 | 0-5% |
| x = 4 | 22-32% |
| x = 5 | 38-42% |
| x = 6 | 18-28% |
| x = 7 | 0-9% |

### SDS-PAGE:

Since SDS-PAGE is the analytical method described in the Tanaka paper, a brief discussion of results for the presently inventive PEGx-GCSF conjugates, wherein x is from 4 to 8, or PEG[x]-GCSF populations of individual conjugates, wherein [x] is 4 or greater, is presented here for comparative purposes. PEG[x]-GCSF samples were prepared by taking a sample volume containing approximately 3 μg of protein in an Eppendorf tube and diluting 6× with sample buffer with DTT. Samples were loaded into the wells of a Nupage 4-12% Bis-tris gel. The gel was run at 200V for 10 min and 150V for 40 min in SDS-MOPS running buffer using Invitrogen's Xcell Surelock electrophoresis system. Fixing was accomplished with acetic acid/methanol, followed by staining with 0.01% Coomassie in 10% acetic acid, 10% MeOH.

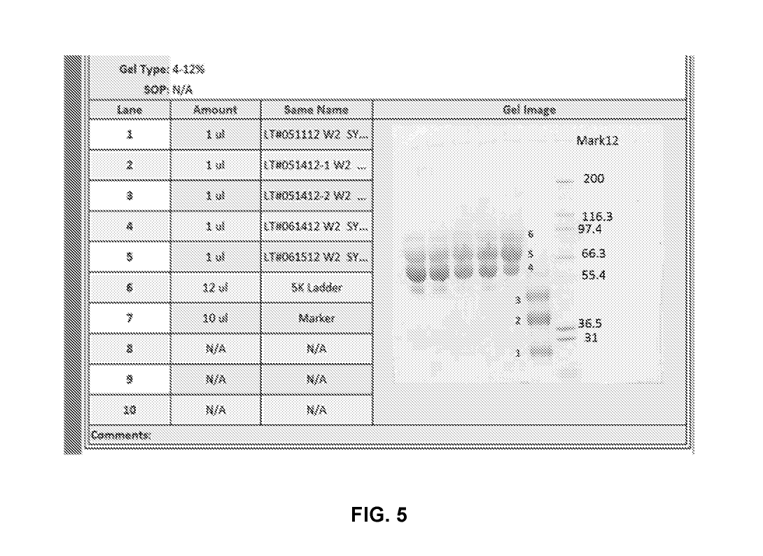

The results of SDS-PAGE analysis are shown in FIG. 5. Results for inventive PEG[x]-GCSF samples appear in Lanes 1-5. Lane 6 contains results for a “5K Ladder” comprised of PEG-GCSF with known PEG/GCSF ratios ranging from 1 to 4. Lane 7 contains protein standard markers. Numbers between lanes 5 and 6 correspond to the expected PEG/GCSF ratios for the various bands present.

It should be noted that the PEG4-GCSF species labeled as having 4 PEGs per GCSF molecule runs at an apparent molecular weight of approximately 55.4 kDa despite the fact that the true average molecular weight is approximately 41 kDa. As discussed for the data reported in the Tanaka paper, the molecular weight of the PEGylated compounds is overestimated when using globular proteins as molecular weight markers. Quantification of bands can be done by densitometry, similar to the procedure used by Tanaka. However, the rapid, automated quantification feature offered by the Bioanalyzer is used in preference to the more laborious, tedious quantification procedure needed for SDS-PAGE, and the results obtained by the two techniques are similar.

Based on a comparison of these results with the information in the Tanaka and Satake-Ishikawa papers, the extent of PEGylation is substantially lower in the previously described compositions, as compared with the extent of PEGylation in the inventive PEGx-GCSF conjugates and PEG[x]-GCSF conjugate mixtures. Comparison with the densitometric scans in FIG. 1 of the Satake-Ishikawa paper shows that Satake-Ishikawa's reaction of SS-PEG with GCSF at PEG/protein ratios of 1, 5, 10, and 50 results in profiles that consist of mono-PEG; mono-PEG+di-PEG; mono-PEG+di-PEG+tri-PEG; to di/tri/tetra-PEG mixtures; but predominantly a mixture of mono- and di-PEGylated GCSF.

The work in the Tanaka paper utilizes GCSF modified with “PEG2” which is understood by those of skill in the art to be 2,4-bis (O-methoxypolyethylene glycol)-6-chloro-s-triazine (activated PEG 10,000; also called PEG2) as described in the Satake-Ishikawa paper. This is a substantially different chemistry than the SC-PEG that is preferably used in the inventive process. The Tanaka paper states that PEG2 (average molecular weight 10,000) was used for PEGylation of GCSF, and the resulting molecular weight was about 45 kDa distributed among 30 kDa, 40 kDa, and 66 kDa. This is consistent with modification by one, two, and three PEG's, respectively, with an average of 2.

Thus, the conjugates prepared by the methods disclosed by either of the Tanaka and Satake-Ishikawa papers result in a mixture of conjugates characterized by having a much lower ratio of PEGgylation than is present in the inventive PEGx-GCSF conjugates, wherein x is from 4 to 8, or PEG[x]-GCSF populations of individual conjugates, wherein [x] is 4, 5, 6, 7 or 8 or greater.

### Sites of PEGylation in the PEGx-GCSF Conjugates

All numerical references to sites on a GCSF molecule in the following discussion correspond to the amino acid sequence shown in FIG. 1, i.e. SEQ ID NO: 1.

A sample of a conjugate mixture of PEG[x]-GCSF having [x] of 4 or greater in accordance with an embodiment of the present invention was digested with endoproteinase Glu-C, which is specific for the carboxy side of acidic residues glutamate and aspartate. Glu-C fragment 1-20, which contains the N-terminus and lysine-17, and Glu-C fragment 35-47, which contains lysine-35 and lysine-41, were not found in the non-PEGylated region of the Glu-C peptide map, consistent with their being PEGylated. In contrast, Glu-C fragment 21-34, which contains lysine-24, revealed no PEGylation at that residue, suggesting complete absence or trace amounts below the level of detection. This data, taken together with the extent of PEGylation data indicating that an average of 4 to 6 PEGs are present on each molecule of GCSF, is consistent with extensive PEGylation of the inventive conjugates at the N-terminus and the lysines at positions 17, 35, and 41 of GCSF. This data meets with expectations in that the N-terminus and lysines at positions 17, 35, and 41 are highly exposed and, consequently, reactive with electrophilic reagents such as SC-PEG, while the lysine at position 24 is relatively buried in the 3-dimensional conformation of the protein and, consequently exhibits highly limited reactivity with SC-PEG Since the evidence from the Bioanalyzer and SDS-PAGE experiments, described above, is that as many as 7 PEGs may be attached to GCSF according to the invention and the average number of PEGs attached to GCSF is closer to 5 than 4, the position of attachment of the remaining 3 PEGs is believed to be on the imidazole groups of Histidine residues, which exist at positions 44, 53, 80, 157, and 171, with preferential modification governed by relative degrees of exposure and local electronic circumstances of the individual Histidine residues.

### Example 3

### In vitro Study Results

This Example compares the cell proliferation activity of the inventive PEG[x]-GCSF vs. that of NEULASTA® with respect to (i) certain cancer cells and (ii) normal bone marrow progenitor cells.

The standard biological screening tests for evaluating Growth Factors include the use of cell lines developed specifically for the analysis and pharmaceutical lot release of GCSF products. The murine M-NSF-60 cell line was developed and is currently in widespread use for the pharmaceutical release testing of GCSF proteins (Mire-Sluiset. Al., Pharm. Pharmacol. Commun. 5, 45-49; Shirafuji N1, Exp Hematol. 1989 Feb;17(2):116-9).

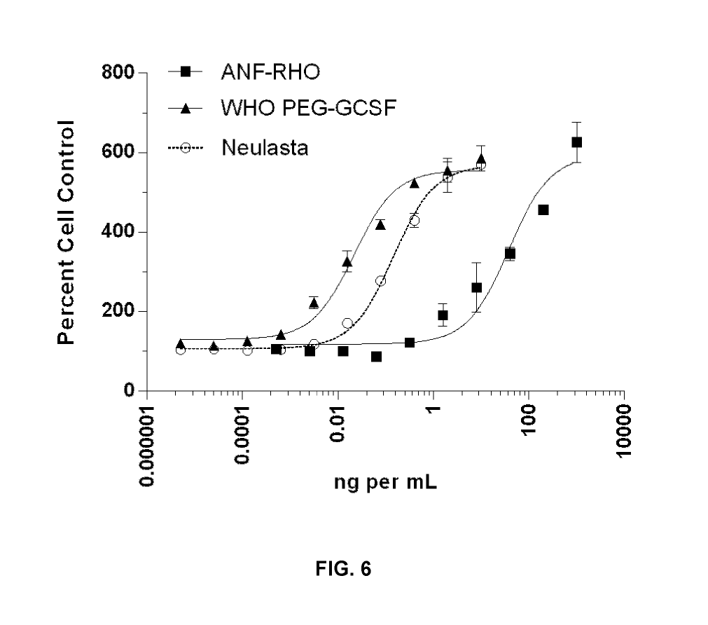

A sample of PEG[x]-GCSF prepared in accordance with Example 1, above, was analyzed in a bioassay in parallel with the International Standard of PEG-GCSF from the World Health Organization (WHO STD) and commercial formulation of Amgen (NEULASTA (ID) using M-NFS-60 cells that were commercially obtained from ATCC and serially passaged twice weekly for a minimum of 20 passages in the presence of 62 ng per mL of recombinant GCSF (r-GCSF). Cell viability was monitored via trypan blue dye exclusion to ensure culture viability remained>95%. On Day 1, cells were counted and cell density was adjusted to 5×10⁵ cells per mL in growth media (RPMI 1640 supplemented with 10% FBS, 0.05 mM BME, 1× Penicillin/Streptomycin, and 62 ng per mL human r-GCSF). Twenty four hours later, cell number was determined by trypan blue exclusion, and cells were collected by centrifugation and suspended at 5×10⁵ cells per mL in assay medium (RPMI 1640 supplemented with 10% Fetal Bovine Serum (FBS), 0.05mM BME, 1× Penicillin/Streptomycin), and returned to 37° C. for 24 hours. On day 3, cells were counted and density was adjusted to 2×10⁵ cells per mL in assay, and 0.1 mL per well of cell suspension was distributed to 96-well plates. Serial 5-fold dilutions at 2× assay concentration of test material in assay medium were prepared and 0.1 ml added. After sample addition, plates were returned to 37° C. incubator for 48 hours. On day 5, 0.04 mL of Promega Cell Titer Aqueous One (Promega, Madison, Wis.) was added to each well and incubated at 37° C. After 4 hours of incubation, optical density (OD) at 490 nM was measured and data were exported to a Microsoft Excel (MSXL) Workbook File. The OD of treated cells were normalized to the OD of untreated control wells and expressed as a percentage of control. Potency estimations were made by fitting data to a three parameter logistic curve fit model (FIG. 6). Representative values of EC₅₀, 95% confidence intervals, along with goodness of fit from FIG. 6 are shown in Table 2 immediately below. The “Relative Potency” column in Table 2 represents the fold difference between the inventive PEG[x]-GCSF (labeled “ANF-Rho” in the Table) and WHO PEG-GCSF or NEULASTA®.

TABLE 2

| | EC₅₀ | 95% Confidence | | Relative |
|---|---|---|---|---|
| | ng per mL | Intervals | r² | Potency |
| ANF-RHO | 38.3 | 18-79 | 0.93 | 1 |
| WHO PEG-GCSF | 0.02 | 0.01-0.03 | 0.98 | 1915 |
| Neulasta | 0.15 | 0.11-0.19 | 0.99 | 255 |

As shown in FIG. 6 and Table 2, WHO STD PEG-GCSF and NEULASTA® demonstrated sub-nanogram potency values of 0.15 and 0.02 ng, respectively. In contrast, the EC₅₀ value of the inventive PEG[x]-GCSF (labeled “ANF-Rho” in FIG. 6 and Table 2) was estimated to be 255 to 1955-fold less potent, with an EC₅₀ value of about 38 ng per mL. All three preparations of PEG-GCSF were able to cause cell proliferation equally at their respective maximum treatment concentrations.

For purposes of comparison with the aforementioned study conducted using murine cancer cell lines, the effect of the inventive PEG[x]-GCSF was also measured on normal human cells. Specifically, hematopoietic stem cells were treated with either NEULASTA® or the inventive PEG[x]-GCSF, and the differentiation and proliferation of CD34⁽⁺⁾ stem cells into mature neutrophils was quantified by flow cytometry, according to the following procedure.

Cryopreserved, bone marrow CD34+ cells and CFC kit were purchased from Stem Cell Technologies (Vancouver, BC, Canada), and the cells were stored at −135° C. until time of assay. Iscove's Modification of Dulbecco's Medium (IMDM) (Life Technologies, Grand Island, N.Y.) supplemented with 10% FBS was warmed to 37° C. Cells were thawed by placing the vial in a 37° C. bath, and viability was immediately determined on a 0.01 mL sample. Cells were transferred to a sterile 50 ml tube containing 300 μg of Dnase I (Life Technologies). Cells were continued to be slowly warmed by gently swirling the tube and adding warm medium until the final volume was 20 ml. The suspension was mixed by gentle inversion. Tube was then centrifuged in a swinging bucket rotor at 200g (840rpm, R&D lab, Beckman JS4.2) at room temperature for 15 minutes. Supernatant was carefully removed with a pipette, leaving a small amount of medium. Cell pellet was then suspended in remaining medium and then 20 mL of medium was added and cells were mixed by gentle inversion. After this step was then repeated once again, cell pellet was then suspended in 1 mL of medium so that cell density was 1.5×10⁵ cells per ml.

MethoCult medium (Stemcell Technologies, Vancouver, BC, Canada) was thawed overnight at 4° C. and then brought to 25° C. just before assay start. NEULASTA® and inventive PEG[x]-GCSF were then added to a final assay concentration of 50 ng per mL and gently mixed by vortex. Two 35 mm culture dishes with lids inside were placed in a 100 mm petri dish with lid. A third 35 mm culture dish without a lid, containing water, was added to maintain proper humidity level. Cells were diluted in IMDM supplemented with 2% FBS to final concentration of 5×10⁵ cells per mL. 0.3 ml of diluted cells was added to 3 ml MethoCult tube and gently mixed by vortex. Mixture was allowed to stand for at least 5 minutes to allow the bubbles to rise to the top. A sterile 16-gauge blunt-end needle was attached to a sterile 3 ml syringe. Air was expelled from the syringe, and then the needle was placed below the surface of the solution and approximately 1 ml was drawn into syringe. The plunger was gently depressed to expel the expel medium completely and repeated until no air space was visible. MethoCult mixture containing cells was drawn into the syringe and dispensed at a volume of 1.1 ml into each 35mm dish without touching the syringe to the dish. Medium was evenly distributed across the surface of each 35 mm dish by gently tilting and rotating the dish to allow the medium to attach to the wall of the dish on all sides, while ensuring no medium touched the lid. The contents of the culture dishes were placed into the 100 mm dish, adding about 3 ml of sterile water to the uncovered 35 mm dish and incubated at 37° C. in 5% CO₂ with >95% humidity for 14 days. Cells were recovered from the methylcellulose matrix by adding 1 ml of IMDM supplemented with 2% FBS medium to each well and pipetted up and down to thoroughly mix. The entire mixture was transferred to a 15 mL tube, and 11 ml of medium was added. Cells were pelleted at 600×g for 10 minutes, and media supernatant was aspirated. Cell pellet was suspended in remaining medium, and cell number was determined by trypan blue exclusion.

Cell pellets were washed twice in 320 μl of cold Stain Buffer (BD Pharmingen, San Jose, Calif.) by centrifuging at 300×g for 5 minutes. Cells were fixed by the addition of 50 μl of Cytofix (BD Pharmingen) to each pellet and resuspended by gently vortexing. Cells were incubated for 20 minutes at 4° C. and then centrifuged at 300×g for 5 minutes. Cells were then washed with 1 ml of stain buffer for a total of three times. Cells were stained with antibodies anti-CD66b and CD-14 directly conjugated with Fluorescein isothiocyanate (FITC) or phycoerythrin (PE). Staining specificity was ensured by using directly conjugated isotype control antibodies. Flow cytometry and data acquisition then were performed.

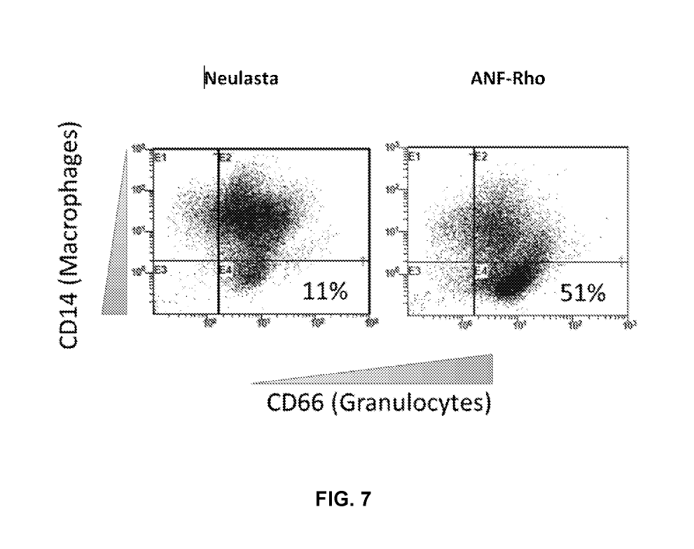

FIG. 7 shows quadrant gating of bivariate plots of fluorescent intensity of CD66 and CD14. CD66(+), CD14(−) cells are indicative of granulocytes (Gate E4), while CD66(−) cells, CD14(+) cells (Gate E1) are characteristic of macrophages. Cells which stain positive for both these antigens appear in Gate E2. NEULASTA® treatment (left panel of FIG. 7) resulted in 11% of the cells staining positive for CD66, while treatment with the inventive PEG-GCSF conjugates (right panel of FIG. 7) resulted in 51% of the cells staining positive for CD66. The fraction of granulocytes was therefore 4.6 times higher in the inventive-conjugate-treated sample as compared to the sample treated with the same concentration of NEULASTA

These results demonstrate that, in contrast to the data in the M-NFS-60 bioassay discussed above (which uses a cell line derived from a myelogenous leukemia, and wherein the inventive PEG[x]-GCSF was much less potent than currently available products), the inventive PEG[x]-GCSF has approximately three orders of magnitude greater potency than NEULASTA® in causing differentiation and proliferation of normal bone marrow cells into granulocytes. When the respective potency data for cancer cell lines and normal cells is taken together, the inventive PEG[x]-GCSF conjugates would be expected to have a larger clinical therapeutic window compared to filgrastim and PEG-filgrastim in causing proliferation of normal bone marrow cells without exacerbating tumor growth of certain cancer types.

Thus, when compared with NEULASTA®, the inventive PEG[x]-GCSF surprisingly has much more of the desired activity of stimulating proliferation of bone marrow cells, while much less of the undesirable activity of causing proliferation of cancer cells.

### Example 4

### Animal Study Results

This Example presents the results of in vivo pharmacokinetic and pharmacodynamic studies in rats, demonstrating the superior ability of the inventive PEG[x]-GCSF to stimulate neutrophil production, when compared to NEULASTA®.

The pharmacokinetics and pharmacodynamics of three lots of the inventive PEG[x]-GCSF, prepared in accordance with Example 1, above, were compared with NEULASTA® after a single subcutaneous (SC) dose to rats. The NEULASTA® was obtained commercially. In addition, formulation buffer used to prepare the proper dosages was included as a negative control in the study.

Ninety-six male 10-and-a-half week old Sprague Dawley (SD) rats were administered 90 milligrams per kilogram of the cancer chemotherapeutic cyclophosphamide on Day −1 by intraperitoneal (IP) injection in order to induce neutropenia. The respective test articles were administered on Day 1 by subcutaneous (SC) injection. Individual doses were calculated based on body weights taken on Day −1 and Day 1 prior to administration of cyclophosphamide and the test articles, respectively. All test article solutions were allowed to reach ambient temperature and were gently inverted and swirled prior to administration. All formulations were clear solutions. The cyclophosphamide was reconstituted in Sterile Water for Injection and was sonicated to provide a clear solution. Three separate lots of the inventive PEG[x]-GCSF were administered at 25, 50 and 100 μg/kg. NEULASTA® was administered at 100 μg/kg. The IP and SC doses were administered by syringe and needle in the abdominal and shaved mid-scapular regions, respectively.

Blood (approximately 0.8 mL) was collected from a jugular vein via syringe and needle and transferred into tubes containing K₂ EDTA anticoagulant pre-dose and at 12, 24, 48, 72, 96, 120, 144, 168, 192, 216, 240, 264, and 288 hours post-dose. Blood was collected from 2 animals/group pre-dose and from 4 animals/group/time point for all post-dose collections. The total blood sample volume for each animal was within the approved limits of the Institutional Animal Care and Use Committee (IACUC).

Each blood sample was divided, with approximately 400 μL blood being transferred to a new tube and centrifuged for plasma. Blood samples were maintained on wet ice prior to centrifugation. Centrifugation began within 1 hour of collection. Plasma was transferred into screw-top cryovials and placed on dry ice prior to storage at approximately −70° C. Absolute Neutrophil Counts (ANC) were determined.

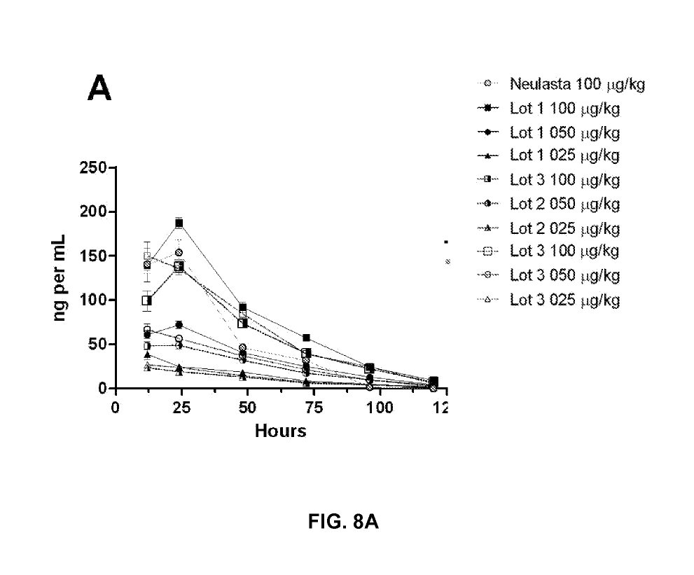

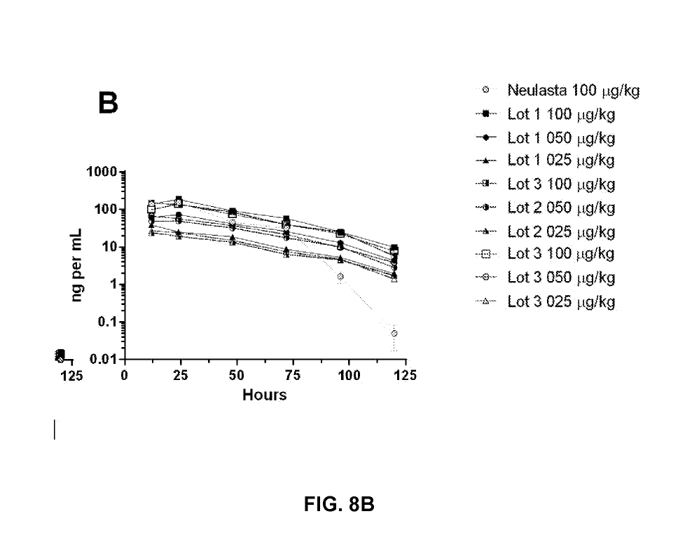

Human GCSF plasma levels then were determined using a commercially purchased ELISA kit according to manufacturer's instructions. (Catalog No. DCS50, RnD Systems, Minneapolis, Minn.). The plasma concentration of inventive PEG[x]-GCSF batches and NEULASTA® as a function of time post administration are shown in FIG. 8. (Data sets corresponding to the inventive PEG[x]-GCSF are labeled as “Lot 1,” “Lot 2,” and “Lot 3,” respectively.)

Statistical analysis: ANC (expressed as 10³ cells per mL) and GCSF plasma levels (expressed as ng per mL) were collated using Excel (Microsoft, Redmond Wash.). Data were visualized as ANC or GCSF concentration as a function of time post-administration using Prism Graphpad (Graphpad Software, La Jolla, Calif.). Area under the curve, one-phase decay, and linear regression analysis for both GCSF and ANC were performed according to software instructions. Analysis of variance (ANOVA) with a Dunnet's post hoc test was performed to demonstrate significant differences (p<0.05) between treatment groups.

FIG. 8A shows that the levels of both PEGylated GCSF molecules peak by <24 hours post administration and then drop off relatively quickly. However, when concentrations are visualized on a log scale in FIG. 8B, the differences between the inventive conjugates and NEULASTA® become apparent. The concentration of NEULASTA® at 100 μg/kg drops approximately 3 orders of magnitude after 72 hours. This is in contrast to the inventive conjugates, which show a slow, somewhat steady, decrease in plasma concentration during the course of the study and remain up to 20-100 times the level of NEULASTA® after 5 days after administration.

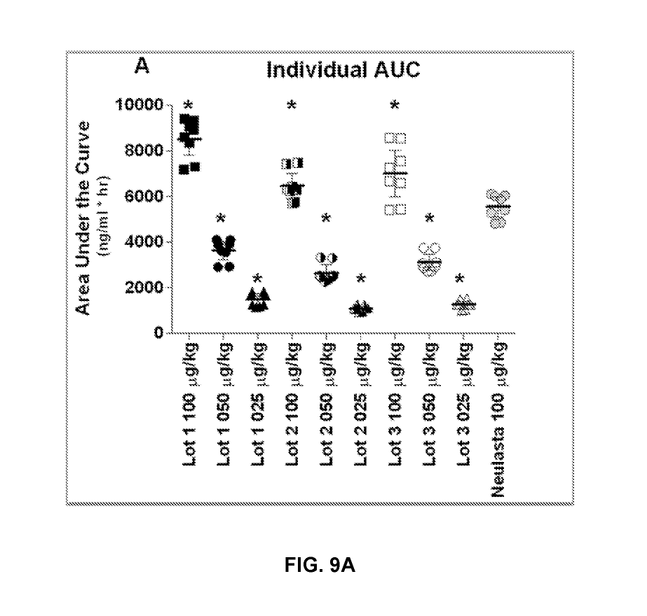

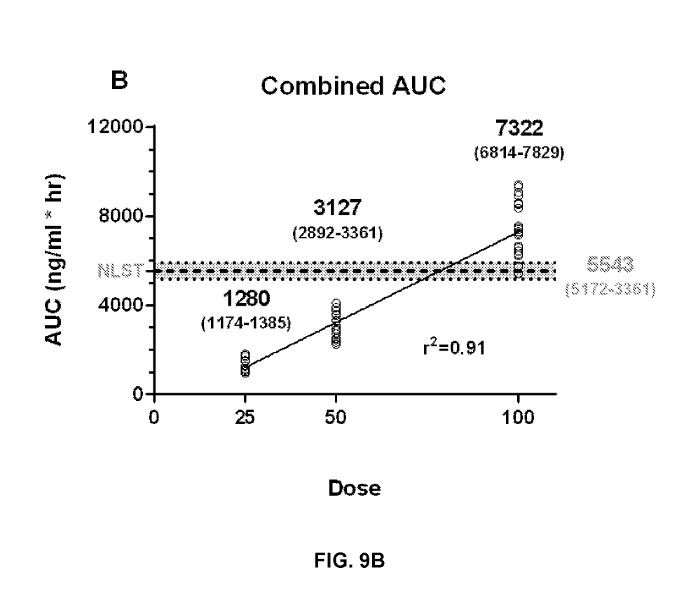

In an effort to further evaluate the differences in plasma levels of treatment groups, pharmacokinetics using “area under the curve” analysis (AUC) was used to quantify the plasma exposure of the inventive conjugates and NEULASTA®. FIG. 9A summarizes the AUC values for each inventive conjugate lot and dosage, as well as NEULASTA®. Each lot of the inventive conjugate dosed at 100 μg/kg resulted in significantly greater plasma concentration as compared to the equal dose of NEULASTA®. Inventive PEG[x]-GCSF plasma levels from animals dosed with 25 or 50 μg/kg were significantly less than the GCSF levels in plasma from NEULASTA® dosed rats (since the NEULASTA® dose was 100 μg/kg). To further characterize the relationship between dose and plasma exposure, the AUC values of each inventive conjugate lot were pooled according to dose and subjected to linear regression analysis (FIG. 9B). The pooled AUC of 7322 ng per ml*hour for 100 μg/kg dosage of inventive PEG[x]-GCSF was significantly higher than 5543 ng per ml*hour calculated for NEULASTA®. Inventive conjugate dosages of 50 and 25 μg/kg yielded significantly lower AUC values of 3127 and 1280, than NEULASTA®, respectively (which, again, is not unexpected, since the NEULASTA® dose was 100 μg/kg). There is a linear relationship between dosage and plasma AUC in this study (r²=0.91). Further, when the individual 100 μg/kg inventive conjugate AUC values are compared to the mean and 95% confidence interval from the rats administered NEULASTA®, 22 of the 24 rats achieved greater plasma exposure at the same dose.

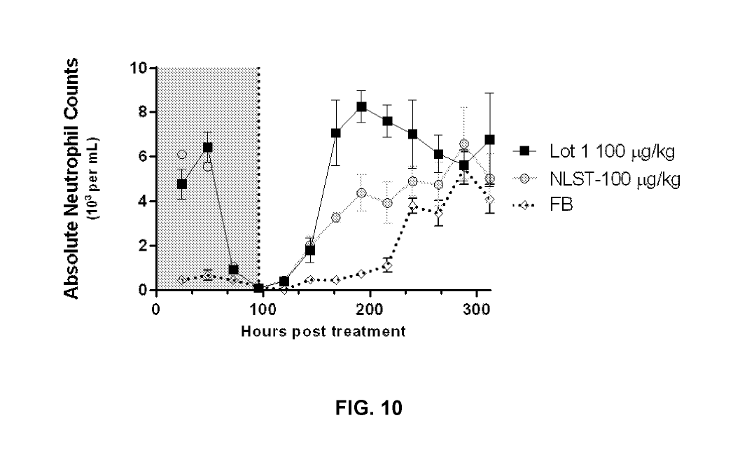

FIG. 10 shows a representative graph of absolute neutrophil count (ANC) as a function of time from rats dosed with inventive PEG[x]-GCSF, NEULASTA® (NLST), or formulation buffer (FB). Two points have to be considered: first, the gradual rise of neutrophil levels from the rats dosed with FB as the innate proliferation response recovers after cyclophosphamide treatment; and second, the sharp increase in ANC levels that immediately follows administration of either PEGylated test article. This second point is attributed to the GCSF-mediated release of pre-formed immature neutrophils from bone marrow and other compartments (shaded area); i.e., “stem cell reservoir mobilization.” Therefore, to accurately compare de novo proliferation that is mediated by inventive conjugate or NEULASTA®, the AUC analysis considered only the ANC levels after 96 hours; data falling within the shaded area of FIG. 10 were not included in ANC analysis. Comparison of ANC counts between ˜125 and 250 hours showed higher ANC counts with inventive PEG[x]-GCSF as compared with NEULASTA® at equivalent dosage levels.

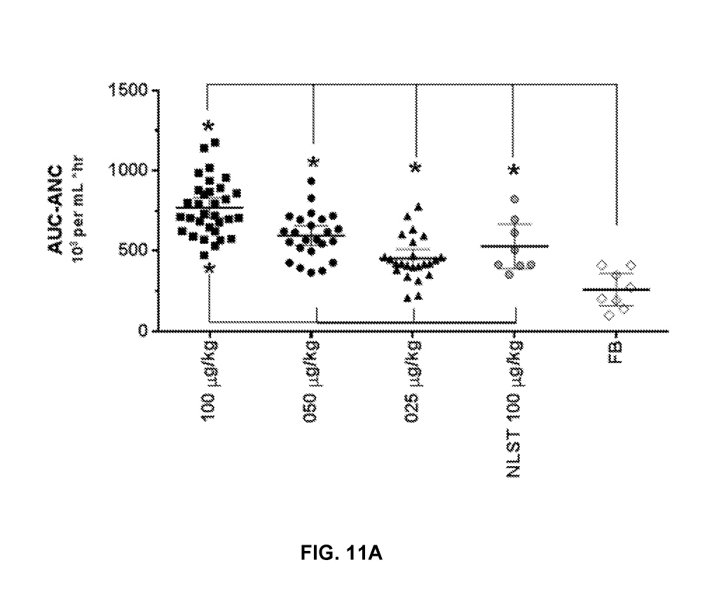

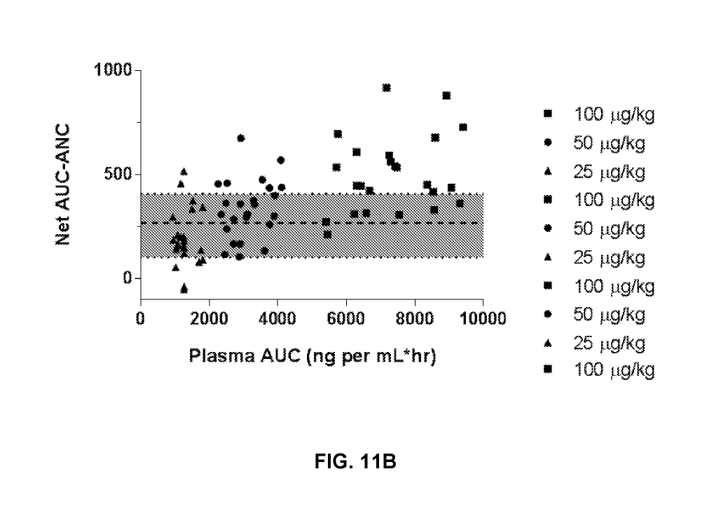

To better analyze potency differences, the AUC of the absolute neutrophil counts (AUC-ANC) over the course of the de novo proliferation period was evaluated with three unique lots of inventive PEG[x]-GCSF at three separate concentrations (25, 50 and 100 μg/kg). In this way, the level and duration of neutrophil production can be quantified. FIG. 11A summarizes the AUC of the absolute neutrophil counts from inventive conjugate and NEULASTA® treated neutropenic rats. When compared to the formulation buffer, both NEULASTA® and the inventive PEG[x]-GCSF at each dosage were able to significantly increase neutrophil proliferation. Furthermore, when compared to NEULASTA® dosed at 100 μg/kg, inventive PEG[x]-GCSF at the 50 and 25 μg/kg level achieved similar neutrophil proliferation and, in the case of the 50 μg/kg dose, slightly increased neutrophil proliferation. Additionally, inventive PEG[x]-GCSF at the same 100 μg/kg dose as NEULASTA®, achieved significantly higher neutrophil levels. A linear regression plot was also produced using the same data sets to further illustrate the pharmacodynamic properties of inventive PEG[x]-GCSF as compared to NLST (shaded area of FIG. 11B).

If the pharmacokinetics (FIG. 9) and pharmacodynamics (FIG. 10) data are taken together, the inventive PEG[x]-GCSF presents a profile different than NEULASTA®. Dosages of 25 and 50 μg/kg of the inventive PEG[x]-GCSF gave significantly lower plasma levels as compared to 100μg/kg NEULASTA®. The plasma GCSF AUC of 25 and 50 μg/kg was 4.3 and 1.8 fold lower compared to 100 μg/kg NEULASTA®, with peak levels 7.2 and 2.9 times respectively lower. However, the lower doses (25 and 50 μg/kg) of inventive conjugate resulted in equivalent neutrophil production in vivo as compared with 100 μg/kg of NEULASTA®. Coupled with the in vitro CD34(+) progenitor results presented in Example 3, which show that an equal dose of inventive conjugate could produce greater than 4-fold more granulocytes than NEULASTA®, these data suggest that the PEG[x]-GCSF is more potent than NEULASTA® in neutrophil production, and has a significantly longer circulating half-life. This suggests the following advantages of the inventive formulations:
Unlike currently commercially available formulations such as NEULASTA®, the inventive PEG[x]-GCSF may be administered and, if necessary, re-administration at current or increased dosage may be considered, at any time during chemotherapy. The rise in ANC after administration of the inventive PEG[x]-GCSF is slower than that attributable to NEULASTA® over the 7-day period following chemotherapy—a critical time when the risk of severe neutropenia is highest. This is because NEULASTA® apparently mobilizes the majority of the stem cell pool (with the result of rapidly producing neutrophils), and therefore the dosing instructions for NEULASTA® call for a delay of 14 days before administering NEULASTA® again—to allow for the stem cells to regenerate themselves. In contrast, the inventive PEG[x]-GCSF seems to mobilize only a smaller part of the stem cell pool (resulting in a more gradual and more prolonged production of neutrophils). For this reason, the 14-day precaution is not necessary in the case of the inventive formulations, based on the slower and more persistent increase of ANC as demonstrated above. Moreover, even without such “repeat dosing,” the action of the inventive formulations keeps ANCs from dropping to neutropenic levels (<2.0×10e5/L). Clinicians typically monitor the response to chemotherapy during the 21-day cycle period. Thus, the inventive PEG[x]-GCSF can be used to support chemo dose-intensification at any time throughout such 21-day cycle if the desired reduction in tumor growth is not observed.
The inventive PEG[x]-GCSF offers an improved quality of life for patients, in that they may be treated with a lower, but equally or more effective, dosage of inventive PEG[x]-GCSF, and/or with less frequent dosing, as compared to NEULASTA®, resulting in, e.g., reduced bone pain.

### Example 5

### Clinical Study (Bone Pain)

In a number of trials of non-PEGylated and PEGylated GCSF, bone pain is the most significant and injurious adverse event (Renwick et. al., 2009). There have been several types of therapeutic interventions which have attempted to prevent and treat GCSF treatment mediated bone pain. Acetaminophen, non steroidal anti-inflammatory drugs, antihistamines and opioids have been employed in attempts to alleviate the pain, with varying degrees of success (Kirsher, 2007, Oagata 2005). One observational, retrospective study showed that no bone pain was observed in 25 patients when the dose of PEG-filgrastim was reduced from 6 mg to 4 mg (Paba et al 2008). Based on the aforementioned preclinical pharmacokinetics and pharmacodynamics, the inventive PEG[x]-GCSF has the potential to be as potent as NEULASTA® at lower doses. It would then follow that, at such lower doses, the inventive conjugates would not cause the bone pain associated with NEULASTA® administration.

A clinical study was conducted, that included ascending single subcutaneous doses of inventive conjugate from 5 to 10 to 20 to 40 to 80 μg/kg weight based dosing regimens. A double-blind, randomized, placebo controlled study also has been conducted to study the safety, tolerability, pharmacokinetics and pharmacodynamics of PEG[x]-GCSF in humans in comparison with NEULASTA®. Volunteers are subcutaneously administered increasing dosages of inventive conjugate with a primary safety endpoint. In addition, among other parameters, bone pain scores are compared to baseline value for both inventive conjugate and NEULASTA®. A visual analog scale (VAS) is used to determine overall pain, along with a specific bone pain questionnaire. Both VAS and bone pain are quantified using a 100 mm horizontal line anchored with pain descriptors and pain indicated by measuring distance from the left hand (no pain) side of the line. Patients who are administered inventive conjugate report less bone pain than patients who are administered NEULASTA®.

We claim:

1. A PEGx-GCSF, wherein x is an integer from 4 to 8.

2. The PEGx-GCSF according to 1, wherein x is 5.

3. The PEGx-GCSF according to claim 1, wherein x is 6.

4. The PEGx-GCSF according to claim 1, wherein x is 7.

5. The PEGx-GCSF according to claim 1, wherein PEG is attached to GCSF through an amine originating from GCSF.

6. The PEGx-GCSF according to claim 1, comprising a non-hydrolyzable linkage.

7. The PEGx-GCSF according to claim 6, wherein the non-hydrolyzable linkage is a urethane linkage.

8. The PEGx-GCSF according to claim 1, wherein GCSF is a protein having an amino acid sequence selected from the group consisting of SEQ ID NO: 1, SEQ ID NO: 3, SEQ ID NO: 4, and functional derivatives and homologs thereof.

9. The PEGx-GCSF according to claim 8, wherein the amino acid sequence is SEQ ID 1, a functional derivative of SEQ. ID 1, or a homolog of SEQ ID 1, wherein the GCSF has a lysine residue at position 17, a lysine residue at position 35, a lysine residue at position 41, a histidine residue at position 44, a histidine residue at position 53, a histidine residue at position 80, a histidine residue at position 157 and a histidine residue at position 171.

10. The PEGx-GCSF according to claim 9, wherein each PEG is attached to GCSF at a position selected from the group consisting of: the N-terminus, the lysine residue at position 17, the lysine residue at position 35, the lysine residue at position 41, the histidine residue at position 44, the histidine residue at position 53, the histidine residue at position 80, the histidine residue at position 157 and the histidine residue at position 171.

11. The PEGx-GCSF according to claim 1, wherein the PEG has an average molecular weight from about 3 to about 15 kDa.

12. The PEGx-GCSF according to claim 11, wherein the PEG has an average molecular weight from about 5 to about 6 kDa.

13. A PEG[x]-GCSF comprising a population of PEGx-GCSF, wherein [x] is an average value of x, and wherein [x] is greater than or equal to about 4.

14. The PEG[x]-GCSF according to claim 13, wherein [x] is from about 4 to about 8.

15. The PEG[x]-GCSF according to claim 13, wherein [x] is from about 4 to about 6.

16. The PEG[x]-GCSF according to claim 13, wherein [x] is from about 5 to about 6.

17. The PEG[x]-GCSF according to claim 13, comprising less than 10% PEGx-GCSF wherein x is from 1 to 3.

18. The PEG[x]-GCSF according to claim 13, comprising at least about 15% PEGx-GCSF wherein x is 4.

19. The PEG[x]-GCSF according to claim 13, comprising at least about 30% PEGx-GCSF wherein x is 5.

20. The PEG[x]-GCSF according to claim 13, comprising at least about 10% PEGx-GCSF wherein x is 6.

21. The PEG[x]-GCSF according to claim 13, comprising less than 15% PEGx-GCSF wherein x is 7.

22. The PEG[x]-GCSF according to claim 13, comprising at least about 15% PEGx-GCSF wherein x is in the range from 6 to 7.

23. The PEG[x]-GCSF according to claim 13, comprising at least about 35% PEGx-GCSF wherein x is in the range from 5 to 7.

24. The PEG[x]-GCSF according to claim 13, wherein PEG is attached to GCSF through an amine originating from GCSF.

25. The PEG[x]-GCSF according to claim 13, wherein the PEGx-GCSF comprises a non-hydrolyzable linkage.

26. The PEG[x]-GCSF according to claim 25, wherein the non-hydrolyzable linkage is a urethane linkage.

27. The PEG[x]-GCSF according to claim 13, wherein GCSF is an amino acid having a sequence selected from the group consisting of SEQ ID NO: 1, SEQ ID NO: 3, SEQ ID No. 4, and functional derivatives and homologs thereof.

28. The PEG[x]-GCSF according to claim 27, wherein the amino acid is SEQ ID NO: 1, a functional derivative of SEQ ID NO: 1, or a homolog of SEQ ID NO: 1, wherein the GCSF has a lysine residue at position 17, a lysine residue at position 35, a lysine residue at position 41, a histidine residue at position 44, a histidine residue at position 53, a histidine residue at position 80, a histidine residue at position 157 and a histidine residue at position 171.

29. The PEG[x]-GCSF according to claim 28, wherein each PEG is attached to GCSF at a position selected from the group consisting of: the N-terminus, the lysine residue at position 17, the lysine residue at position 35, the lysine residue at position 41, the histidine residue at position 44, the histidine residue at position 53, the histidine residue at position 80, the histidine residue at position 157 and the histidine residue at position 171.

30. The PEG[x]-GCSF according to claim 13, wherein the PEG has an average molecular weight from about 3 to about 15 kDa.

31. The PEG[x]-GCSF according to claim 30, wherein the PEG has an average molecular weight from about 5 to about 6 kDa.

32. The PEG[x]-GCSF according to claim 13, said population comprising:
from about 0% to about 5% of PEGx-GCSF wherein x is 3;
from about 22% to about 32% of PEGx-GCSF wherein x is 4;
from about 38% to about 42% of PEGx-GCSF wherein x is 5;
from about 18% to about 28% of PEGx-GCSF wherein x is 6; and
from about 0% to about 9% of PEGx-GCSF wherein x is 7.

33. The PEG[x]-GCSF according to claim 13, wherein the PEG is attached to the GCSF through a urethane linkage and wherein the PEG has an average molecular weight molecular weight from about 3 to about 15 kDa.

34. The PEG[x]-GCSF according to claim 33, wherein the PEG has an average molecular weight from about 5 to about 6 kDa.

35. A pharmaceutical formulation comprising a pharmaceutically active amount of PEG[x]-GCSF according to claim 13 and a protein-free carrier.

36. A method of increasing the white blood cell count in a patient in need thereof, comprising administering to said patient a therapeutically effective amount of the pharmaceutical formulation of claim 35.

37. The method according to claim 36, wherein the patient is at risk of or is suffering from neutropenia.

38. The method according to claim 36, wherein the patient has been, or is being treated with, an agent that decreases the patient's white blood cell count.

39. The method according to claim 36, wherein the patient has decreased endogenous levels of GCSF.

40. The method according to claim 36, wherein the patient is undergoing radiation treatment.

41. The method according to claim 38, wherein the patient is being treated for cancer.

42. The method according to claim 41, wherein the cancer is a myeloid cancer.

43. The method according to claim 37, wherein the patient is suffering from severe chronic neutropenia or severe congenital neutropenia or severe combined neutropenia.

44. The method according to claim 36, wherein the patient is treated prior to an autologous stem cell transplant.

45. A method of treating severe sepsis or septic shock in a patient in need thereof, the method comprising administering to said patient a therapeutically effective amount of the pharmaceutical formulation of claim 35.

46. A method for preparing the PEGx-GCSF according to claim 1, said method comprising the steps of:
(a) obtaining a GCSF solution having a concentration of about 5.0 mg/ml;
(b) combining the GCSF solution with PEG, wherein PEG is present in a molar amount of about 65 to about 75 times the molar amount of the GCSF;
(c) allowing sufficient time for the GCSF and PEG to react to produce PEGx-GCSF;
(d) adding hydroxylamine in an amount sufficient to react with residual PEG;
(e) isolating PEG[x]-GCSF from unreacted PEG, N-hydroxysuccinimide and hydroxylamine; and
(f) isolating PEGx-GCSF.

47. The method according to claim 46, wherein the GCSF solution of about 5.0 mg/ml is obtained by a step of concentrating a solution of GCSF.

48. The method according to claim 46, further comprising the step (g) of concentrating the isolated PEGx-GCSF in solution to about 5.5 to 6 mg/ml.

49. The method according to claim 47, wherein said concentrating step is achieved by membrane diafiltration.

50. The method according to claim 48, wherein said concentrating step is achieved by membrane diafiltration.

51. The method according to claim 46, wherein the pH is maintained at about 7.75 during steps (a) through (e).

52. The method according to claim 46, wherein the temperature is maintained at room temperature throughout the method.

53. The method according to claim 46, wherein steps (b) and (c) are conducted for about 1 hour.

54. The method according to claim 46, wherein step (d) is conducted for about 2 hours.

55. A method for preparing PEG[x]-GCSF according to claim 13, said method comprising the steps of:
(a) obtaining a GCSF solution having a concentration of about 5.0 mg/ml;
(b) combining the GCSF solution with PEG, wherein PEG is present in a molar amount of about 65 to about 75 times the molar amount of the GCSF;
(c) allowing sufficient time for the GCSF and PEG to react to produce PEGx-GCSF;
(d) adding hydroxylamine in an amount sufficient to react with residual PEG; and
(e) isolating PEGx-GCSF from unreacted PEG, N-hydroxysuccinimide and hydroxylamine.

56. The method according to claim 55, wherein the GCSF solution of about 5.0 mg/ml is obtained by a step of concentrating a solution of GCSF.

57. The method according to claim 55, further comprising the step (f) of concentrating the isolated PEGx-GCSF in solution to about 5.5 to 6 mg/ml.

58. The method according to claim 56, wherein said concentrating step is achieved by membrane diafiltration.

59. The method according to claim 57, wherein said concentrating step is achieved by membrane diafiltration.

60. The method according to claim 55, wherein the pH is maintained at about 7.75 during steps (a) through (e).

61. The method according to claim 55, wherein the temperature is maintained at room temperature throughout the method.

62. The method according to claim 55, wherein steps (b) and (c) are conducted for about 1 hour.

63. The method according to claim 55, wherein step (d) is conducted for about 2 hours.
